(19) United States
(12) Patent Application Publication
(10) Pub. No.: US 2003/0027259 A1
(43) Pub. Date: Feb. 6, 2003

(54) Novel ABCG4 transporter and uses thereof

(75) Inventors: Hongyun Chen, Vancouver (CA); Stephane Le Bihan, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Active Pass Pharmaceuticals, Inc., Vancouver (CA)

(21) Appl. No.: 10/090,455

(22) Filed: Mar. 1, 2002

Related U.S. Application Data
(60) Provisional application No. 60/272,886, filed on Mar. 2, 2001.
(60) Provisional application No. 60/309,262, filed on Jul. 31, 2001.
(60) Provisional application No. 60/316,339, filed on Aug. 29, 2001.

(51) Int. Cl. C12Q 1/68; C07H 21/04; C12P 21/02; C12N 5/06

(52) U.S. Cl. 435/69.1; 435/320.1; 435/325; 435/6; 530/350; 536/23.5

(57) ABSTRACT

The invention provides isolated nucleic acid molecules, designated ABCG4 transporter nucleic acid molecules, which encode novel ABC transporter family members. The invention also provides antisense nucleic acid molecules, recombinant expression vectors containing ABCG4 transporter nucleic acid molecules, host cells into which the expression vectors have been introduced, and non-human transgenic animals in which an ABCG4 transporter gene has been introduced or disrupted. The invention further provides isolated ABCG4 transporter proteins, fusion proteins, antigenic peptides, anti-ABCG4 transporter antibodies, and screening assays for ABCG4 transporter modulators. Diagnostic and therapeutic methods utilizing compositions of the invention are also provided.

### BACKGROUND OF THE INVENTION

1. Field of the Invention

The invention relates generally to the field of an ATP Binding Cassette (ABC) transporter family, and more specifically to a novel member of the family and the use thereof.

2. Description of the Related Art

ABC transporter proteins represent a large superfamily of proteins with conserved features in both prokaryotes and eukaryotes. ABC transporters catalyze ATP-dependent transport of endogenous or exogenous substrates across biological membranes (Borst, P., (1997) Seminar in Cancer Biology 8:131-213) and/or allosterically modify the function of heterologous proteins (Higgins C F, 1995, Cell 82:693-696). Several ABC transporters have been associated with clinically relevant phenotypes including the phenomenon of multidrug resistance (Ambudkar S. V. et al., (1999), Annu. Rev. Toxicol., 39:361-398), cystic fibrosis (Riordin J R et al., (1989) Science 245:1066-1073), atherosclerosis (Brooks-Wilson A et al., (1999) Nature Genetics 22:336-345), hyperinsulinemic hypoglycemia (Thomas P M et al., (1995) Science 268:46-429), macular degeneration (Allikmets R et al., (1997) Science 277:1805-1807), and adrenoleukodystrophy (Mosser J et al, (1993) Nature 361:726-730) to name but a few. There is a need in the art for identification of the full-length nucleotide sequence of the coding region of the ABC transporter genes and translated protein products.

### BRIEF SUMMARY OF THE INVENTION

The present invention is based, at least in part, on the discovery of a novel ATP Binding Cassette (ABC) transporter family member, referred to herein as ABCG4 transporter nucleic acid and protein molecules. The ABCG4 transporter molecules of the present invention are useful as targets for developing modulating agents to regulate a variety of cellular processes, particularly the transport of neurotoxic molecules, e.g., β-amyloid peptide (Aβ), across cell membranes or, e.g., the blood-brain barrier (BBB). Neurotoxic molecules such as β-amyloid peptide are involved in neurological disorders such as Alzheimer's disease (see, e.g., Goate et al. (1991) Nature 349:704; Games et al. (1995) Nature 373:523; and Suzuki et al. (1994) Science 264:1336). Other neurological diseases involving toxic polypeptides include, e.g., prion diseases, Huntington's disease, Parkinson's disease, Amyotrophic Lateral Sclerosis, Spinocerebellar Ataxia, Frontotemporal Dementia, etc. (Hardy et al. (1998) Science 282:1075-1079; Wolozin et al. (2000) Arch. Neurol., 57:793-796). Accordingly, modulation of amyloid-β protein export with a modulator of the human ABCG4 transporter would be expected to modulate amyloid deposition and thus, Alzheimer's disease. In addition, the ABCG4 transporter molecules of the invention are useful as targets for developing modulating agents of multidrug resistance. Moreover, the molecules of the present invention are useful as diagnostic and therapeutic tools.

Accordingly, in one aspect, this invention provides isolated nucleic acid molecules encoding ABCG4 transporter proteins or functional fragments thereof. In one embodiment, the isolated nucleic acid comprises a nucleotide sequence which encodes a polypeptide comprising the amino acid sequence set forth in SEQ ID NOs: 2 or 13 (i.e., human ABCG4 transporter protein). In another embodiment, the isolated nucleic acid comprises a nucleotide sequence which encodes a naturally occurring allelic variant of a polypeptide comprising the amino acid sequence set forth in SEQ ID NOs: 2 or 13, wherein said allelic variant binds to an antibody that selectively binds to the polypeptides of SEQ ID NOs: 2 or 13, and is not the polypeptide within the amino acid sequence of SEQ ID NO: 4 (i.e., a partial human ABCG4 transporter sequence in GenBank AN: CAC17140). In a preferred embodiment, the isolated nucleic acid molecule comprises the nucleotide sequence set forth in SEQ ID NOs: 1, 3, or 12.

In another aspect, this invention provides an isolated nucleic acid molecule comprising a nucleotide sequence that is complementary to the nucleotide sequence described above.

This invention also provides an isolated nucleic acid molecule comprising a nucleic acid sequence encoding an ABCG4 transporter and a nucleotide sequence encoding a heterologous polypeptide.

Another aspect of the invention provides a vector comprising a nucleic acid molecule encoding an ABCG4 transporter or a functional fragment thereof. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing an ABCG4 transporter protein or a functional fragment thereof, by culturing in a suitable medium, a host cell of the invention containing a recombinant expression vector, such that the protein is produced.

In another aspect, the invention also provides an isolated ABCG4 transporter polypeptide or a functional fragment thereof. In one preferred embodiment, the polypeptide comprises the amino acid sequence of SEQ ID NOs: 2 or 13. In another preferred embodiment, the polypeptide comprises a naturally occurring allelic variant of a polypeptide comprising the amino acid sequence set forth in SEQ ID NOs: 2 or 13, wherein said allelic variant binds to an antibody that selectively binds to the polypeptide of SEQ ID NO: 2 or 13, and is not the polypeptide with the amino acid sequence of SEQ ID NO: 4.

The proteins of the present invention or portions thereof, e.g., biologically active portions thereof, can be operatively linked to heterologous amino acid sequences (e.g., a non-ABCG4 transporter polypeptide) at either the amino- or the carboxyl-terminus of the proteins to form fusion proteins. The invention further features antibodies, such as monoclonal or polyclonal antibodies, that specifically bind ABCG4 transporter proteins. In addition, the ABCG4 transporter proteins or functional fragments thereof can be incorporated into pharmaceutical compositions, which optionally include pharmaceutically acceptable carriers.

In another aspect, the present invention provides a method for detecting the presence of an ABCG4 polypeptide or a functional fragment thereof in a biological sample. The method comprises the following two steps: contacting the sample with a compound which selectively binds to the ABCG4 polypeptide or its functional fragment, and detecting the presence of a complex between the compound and the polypeptide or its functional fragment. In a preferred embodiment, the compound that binds to the polypeptide is an antibody. In addition, the invention also provides a kit comprising a compound that selectively binds to an ABCG4 polypeptide or a functional fragment thereof and instructions for use.

In another aspect, the present invention provides a method for detecting the presence of a nucleic acid molecule encoding an ABCG4 transporter or a functional fragment thereof in a biological sample. The method comprises the following two steps: contacting the sample with a nucleic acid probe or primer which selectively hybridizes to the nucleic acid molecule, and detecting the presence of a complex of the nucleic acid molecule and the probe or primer. In one embodiment, the sample comprises mRNA molecules and is contacted with a nucleic acid probe. In addition, the present invention also provides a kit comprising a compound that selectively hybridizes to a nucleic acid molecule encoding an ABCG4 transporter or a functional fragment thereof and instructions for use.

The present invention also provides a method for identifying a compound that binds to an ABCG4 polypeptide or a functional fragment thereof. The method comprises the following two steps: contacting the polypeptide, the functional fragment, or a cell expressing the polypeptide or the functional fragment with a test compound; and determining whether the polypeptide binds to the test compound. The binding of the test compound to the polypeptide may be detected directly, or by a competitive binding assay or an assay for ABCG4 transporter activity.

The present invention further provides methods for modulating the activity of an ABCG4 polypeptide or a functional fragment thereof. One method comprises the following two steps: contacting the polypeptide, the functional fragment, or a cell expressing the polypeptide or its finctional fragment with a compound that binds to the polypeptide in a sufficient concentration to modulate the activity of the polypeptide. In one embodiment, the compound inhibits ABCG4 transporter activity. In another embodiment, the compound modulates the ability of the ABCG4 transporter to allosterically modify the function of other membrane proteins. In yet another embodiment, the compound stimulates ABCG4 transporter activity. In one embodiment, the compound is an antibody that specifically binds to an ABCG4 transporter protein.

Another method for modulating the activity of an ABCG4 polypeptide or a functional fragment thereof is to contact a cell capable of expressing an ABCG4 transporter with an agent that modulates ABCG4 transporter activity such that ABCG4 transporter activity in the cell is modulated. In one embodiment, the agent inhibits ABCG4 transporter activity. In another embodiment, the agent modulates the ability of the ABCG4 transporter to allosterically modify the function of other membrane proteins. In another embodiment, the agent stimulates ABCG4 transporter activity. In one embodiment, the agent is an antibody that specifically binds to an ABCG4 transporter protein. In another embodiment, the agent modulates expression of ABCG4 transporter by modulating transcription of an ABCG4 transporter gene or translation of an ABCG4 transporter mRNA. In yet another embodiment, the agent is a nucleic acid molecule having a nucleotide sequence that is antisense to the coding strand of an ABCG4 transporter mRNA or an ABCG4 transporter gene.

In one embodiment, the methods of the present invention are used to treat a subject having a disorder characterized by aberrant or unwanted ABCG4 transporter protein or nucleic acid expression or activity by administering an agent which is an ABCG4 transporter modulator to the subject. In one embodiment, the ABCG4 transporter modulator is an ABCG4 transporter protein. In another embodiment the ABCG4 transporter modulator is an ABCG4 transporter nucleic acid molecule. In yet another embodiment, the ABCG4 transporter modulator is a polypeptide antibody (or fragment thereof), peptide, peptidomimetic, or other small molecule, e.g. a molecule that is carbohydrate-based, lipid-based, nucleic acid-based, natural organic-based, or synthetically derived organic-based.

In another aspect, the present invention also provides a method for identifying a compound that modulates the activity of an ABCG4 polypeptide or a functional fragment thereof. The method comprises the following two steps: contacting an ABCG4 polypeptide or a functional fragment thereof with a test compound, and determining the effect of the test compound on the activity of the polypeptide or the functional fragment to thereby identify a compound which modulates the activity of the polypeptide or the functional fragment.

In another aspect, the present invention provides a method for detecting an allelic variation of the nucleic acid of SEQ ID NOs: 1 or 12 or an orthologue thereof in a biological sample. The method comprises the following two steps: (a) obtaining from said sample a polynucleotide that hybridizes to the nucleic acid of SEQ ID NOs: 1 or 12 or the orthologue thereof; and (b) determining whether the polynucleotide is identical to a portion, or the full length sequence of SEQ ID NOs: 1 or 12, or the orthologue thereof.

The present invention also provides a composition comprising a pharmaceutically effective amount of the nucleic acid molecule of SEQ ID NOs: 1 or 12, or a functional fragment thereof and a pharmaceutically acceptable carrier. In a related aspect, the present invention provides a composition comprising a pharmaceutically effective amount of an antisense oligonucleotide capable of specifically hybridizing to a portion, or the full length, of the nucleic acid sequence of SEQ ID NOs: 1 or 12 or a functional fragment thereof and a pharmaceutical acceptable carrier.

In another aspect, the present invention provides a method for detecting the presence of ABCG4 transporter activity in a biological sample by contacting the biological sample with an agent capable of detecting an indicator of ABCG4 transporter activity such that the presence of ABCG4 transporter activity is detected in the biological sample.

The present invention also provides a diagnostic assay for identifying the presence or absence of a genetic alteration characterized by at least one of (i) aberrant modification or mutation of a gene encoding an ABCG4 transporter protein; (ii) mis-regulation of the gene; and (iii) aberrant post-translational modification of an ABCG4 transporter protein, wherein a wild-type form of the gene encodes a protein with an ABCG4 transporter activity.

In another aspect the invention provides a method for identifying a compound that binds to or modulates the activity of an ABCG4 transporter protein, by providing an indicator composition comprising an ABCG4 transporter protein having ABCG4 transporter activity, contacting the indicator composition with a test compound, and determining the effect of the test compound on ABCG4 transporter activity in the IT indicator composition to identify a compound that modulates the activity of an ABCG4 transporter protein, e.g., an ABCG4 transporter protein associated with a membrane.

A further embodiment of the current invention provides a transgenic knockout mouse whose genome comprises a homozygous disruption in its endogenous ABCG4 gene, wherein the homozygous disruption prevents the expression of a functional ABCG4 protein, and wherein the homozygous disruption results in the transgenic knockout mouse being sterile.

Another embodiment of the current invention provides a transgenic knockout mouse whose genome comprises a homozygous disruption in its endogenous ABCG4 gene, wherein the homozygous disruption prevents the expression of a functional ABCG4 protein, and wherein the homozygous disruption results in the transgenic knockout mouse suffering from hypo- or hypercholesterolemia compared to a wild type mouse.

Another embodiment of the current invention provides a transgenic knockout mouse whose genome comprises a homozygous disruption in its endogenous ABCG4 gene, wherein the homozygous disruption prevents the expression of a functional ABCG4 protein, and wherein the homozygous disruption results in the transgenic knockout mouse being prone to stroke or arteriosclerosis stroke compared to a wild type mouse.

Another embodiment of the current invention provides a transgenic knockout mouse whose genome comprises a homozygous disruption in its endogenous ABCG4 gene, wherein the homozygous disruption prevents the expression of a functional ABCG4 protein, and wherein the homozygous disruption results in the transgenic knockout mouse having an increased incidence of Neimann-Pick Disease, compared to a wild type mouse.

Another embodiment of the current invention provides a transgenic knockout mouse whose genome comprises a homozygous disruption in its endogenous ABCG4 gene, wherein the homozygous disruption prevents the expression of a functional ABCG4 protein, and wherein the homozygous disruption results in the transgenic knockout mouse having a decreased incidence of Alzheimers disease, compared to a wild-type mouse.

### BRIEF DESCRIPTION OF THE SEVERAL VIEWS OF THE DRAWING(S)

FIG. 1 depicts the cDNA sequence of a human ABCG4 transporter. The nucleotide sequence corresponds to nucleic acids 1 to 3455, which is also represented by SEQ ID NO: 1. The coding region without the 5′ and 3′ untranslated regions of the human ABCG4 transporter gene corresponds to nucleic acids 7-1947 which is represented by SEQ ID NO: 3.

FIG. 2 depicts the cDNA sequence of a human ABCG4 transporter (SEQ ID NO: 1) and its encoded amino acid sequence (SEQ ID NO: 2).

FIG. 3 depicts amino acid sequence of the ABCG4 transporter molecule corresponding to amino acids 1 to 646 which is represented by SEQ ID NO: 2.

FIG. 4 depicts the multiple sequence alignment of the members of the ABCG subfamily (SEQ ID NOs:4-7).

FIG. 5 depicts the sequence alignment of a partial ABCG4 transporter protein in GenBank (AN: CAC17140) (SEQ ID NO:8) and the ABCG4 of the invention (SEQ ID NO: 2).

FIG. 6 shows cellular APP levels from WT6 transiently transfected with a gene encoding β-galactosidase, ABCG4 or one of three ABCG4 mutant proteins. After a 48 hr transfection interval, cells were incubated for 4 hrs and cellular APP was quantitated by Western blot analysis. A representative micrograph of the APP Western blot data is depicted above the corresponding densitometric values. Data are expressed as mean±SD with n=5 and statistical significance determined by ANOVA with Tukey's post hoc test at ***p<0.001.

FIG. 7 shows AP release from WT6 cells transiently transfected with a gene encoding β-galactosidase, ABCG4 or one of three ABCG4 mutant proteins. After a 48 hr transfection interval, cells were incubated for 4 hrs and Aβ release was quantitated by Western blot analysis. A representative micrograph of the Aβ Western blot data is depicted above the corresponding densitometric values. Data are expressed as mean±SD with n=5 and statistical significance determined by ANOVA with Tukey's post hoc test at ***p<0.001.

FIG. 8 shows cellular APP levels from SM4 cells transiently transfected with a gene encoding β-galactosidase, ABCG4 or one of three ABCG4 mutant proteins. After a 48 hr transfection interval, cells were incubated for 16 hrs and cellular APP was quantitated by Western blot analysis. Data are expressed as mean±SD with n=5 and statistical significance determined by ANOVA with Tukey's post hoc test at *p<0.05.

FIG. 9 shows Aβ release from SM4 cells transiently transfected with a gene encoding β-Galactosidase, ABCG4 or one of three ABCG4 mutant proteins. After a 48 hr transfection interval, cells were incubated for 16 hrs and Aβ-40 and Aβ-42 release were quantitated by ELISA. Data are expressed as mean±SD with n=5 and statistical significance determined by ANOVA with Tukey's post hoc test at **p<0.01 and ***p<0.001.

FIG. 10 depicts the cDNA sequence of a human ABCG4.2 transporter. The nucleotide sequence corresponds to nucleic acids 1 to 2687, which is also represented by SEQ ID NO: 12.

FIG. 11 depicts amino acid sequence of the ABCG4.2 transporter molecule corresponding to amino acids 1 to 646 which is represented by SEQ ID NO: 13.

### DETAILED DESCRIPTION OF THE INVENTION

The present invention is based, at least in part, on the discovery of novel ATP Binding Cassette (ABC) transporter family members, referred to herein as ABCG4 transporter nucleic acid and protein molecules. ABC transporter molecules are transmembrane proteins that catalyze ATβ-dependent transport of endogenous or exogenous substrates across biological membranes. ABC transporters have been associated with the transport of polypeptides, e.g., a neurotoxic polypeptide, such as β-amyloid, which is involved in Alzheimer's disease. Other neurological diseases caused by neurotoxic polypeptides include prion diseases, Parkinson's disease, Huntington's disease, etc. (for a review, see Hardy et al. (1998) Science 282: 1075-1079). In particular, ABC transporters are associated with the transport of substrates across the blood-brain-barrier. In addition, ABC transporters are associated with multidrug resistance found in cells especially, e.g, cells that are refractory to cytotoxic anti-cancer drugs (Borst, P. (1997) Sem. Cancer Bio. 8:131-134).

Accordingly, the ABCG4 transporter molecules of the invention are suitable targets for developing novel diagnostic targets and therapeutic agents to control cellular transport in cells of the brain (e.g., neuronal cells) and transport across the blood-brain-barrier. Moreover, the ABCG4 transporter molecules are suitable targets for developing diagnostic targets and therapeutic agents for detecting and/or treating cells or tissues having multidrug resistance, e.g., a cancer.

In particular, the novel human ABCG4 transporter molecules described herein are believed to have one or more of the following finctions and/or applications:

First, ABC transporters expressed in the brain are implicated in the transport of substrates through the blood brain barrier (Schinkel A.H., et al, (1994) Cell, 77, 491) and therefore identification of the sequence of the human ABCG4 transporter described herein affords the development of new strategies for altering the function of the blood brain barrier. Given that many drugs of potential utility in treating diseases of the brain are discarded because they do not enter the brain at therapeutically relevant concentrations, the present invention allows for the development of strategies to assist in the delivery of drugs to the brain.

Second, ABC transporters expressed in the brain (as described in, e.g., U.S. patent application Ser. No. 08/847,616, the text of which is incorporated herein) are potential transporters for the β-amyloid peptide, a peptide whose deposition in senile plaques is a fundamental feature of Alzheimer's disease. Thus, identifying novel transporters that regulate β-amyloid deposition is crucial in developing therapeutic treatment for Alzheimer's disease.

Third, the human homologue of the Drosophila melanogaster white gene has been reported to be associated with mood and panic disorders (Nakamura, M. et al., (1999), Mol Psychiatry, 4, 155-162), and this gene is a member of the superfamily of ABC transporters. Identification of the sequence of human ABC transporter described herein allows for the development of new treatments for mood and panic disorders.

Fourth, ABC transporters have been shown to be involved in the phenomenon of multidrug resistance (Ling, V., (1997) Cancer Chemother Pharmacol 40:S3-S8. The present invention will allow precise determination of the ability of ABCG4 to contribute to the multidrug resistance phenotype and the design of agents capable of ameliorating multidrug resistance using techniques similar to those described by Boer, R., et al. ((1996) European Journal of Cancer, 32A:857-861).

Fifth, the human ABCl protein has been shown to be associated with cholesterol efflux and mutated forms cause Tangier disease and familial high-density lipoprotein deficiency (Brooks-Wilson A. et al, Bodzioch M. et al, Rust S. et al, Nature Genetics, 22, 336-345, 347-351, 352-355 respectively). As the ABCl protein is a homologue of the ABCG4 protein, human ABCG4 may also be found to be a cholesterol transporter. Identification of the sequence of human ABCG4 allows for the development of new treatments for diseases involving cholesterol misregulation.

Sixth, the mouse abc I protein has been implicated in interleukin-1β (IL-1β) secretion from macrophages (Hamon Y. et al, 1997, Blood, 90, 2911-2915). IL-1β is a mediator of inflammatory reactions, and agents able to impair its production or secretion are of potential therapeutic importance. Thus, identification of the related sequence of human ABCG4 allows for the development of new treatments for inflammatory diseases.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics.

For example, the family of ABC transporter proteins comprise at least one “transmembrane domain” and preferably two transmembrane domains. As used herein, the term “transmembrane domain” includes an amino acid sequence of about 18 amino acid residues in length which spans the plasma membrane. More preferably, a transmembrane domain includes about at least 18, 20, 25, 30, 35, 40, or 45 residues or more and spans the plasma membrane. Transmembrane domains are described in, for example, Zagotta W. N. et al, (1996) Annual Rev. Neuronsci. 19: 235-63, the contents of which are incorporated herein by reference. One or more of these transmembrane domains may associate to form a membrane-spanning domain.

Isolated proteins of the present invention, preferably ABCG4 transporter proteins or functional fragment thereof, have an amino acid sequence sufficiently homologous to the full length, or a portion, of the amino acid sequence of SEQ ID NOs: 2 or 13 or are encoded by a nucleotide sequence sufficiently homologous to SEQ ID NOs: 1, 3, or 12. As used herein, the term “sufficiently homologous” in the context of amino acid sequences refers to an amino acid sequence that contains a sufficient or minimum number of identical or equivalent (e.g., an amino acid residue which has a similar side chain) amino acid residues relative to a reference amino acid such that the two sequences share common structural domains or motifs and/or a common functional activity. When used in the context of nucleotide sequences, this term refers to a nucleotide sequence that contains a sufficient or minimum number of identical nucleotide residue relative to a reference nucleotide sequence. For example, sufficiently homologous amino acid sequences typically have at least 50% homology, more preferably 60%, even more preferably 70%-80%, and most preferably 90-95% or higher homology across the amino acid sequences of their shared common domains. Likewise, sufficiently homologous nucleotide sequences generally have at least 50%, more preferably 60%, even more preferably 70%-80%, and most preferably 90-95% or higher sequence identity.

As used interchangeably herein, “ABCG4 transporter activity,” “biological activity of an ABCG4 transporter” or “functional activity of an ABCG4 transporter,” refers to an activity exerted by an ABCG4 transporter protein, polypeptide or nucleic acid molecule on an ABCG4 transporter responsive cell or on an ABCG4 transporter protein substrate, as determined in vivo, or in vitro, according to standard techniques.

Preferably, an ABCG4 transporter activity has the ability to act as an energy-dependent (ATP) molecular pump. In one embodiment, an ABCG4 activity is a direct activity, such as an association with a membrane-associated protein and/or the transport of an endogenous or exogenous substrate across a biological membrane. In another embodiment, the ABCG4 activity is the ability of the polypeptide to allosterically modify the function of other membrane proteins. For example, in some cells, modulation of p-glycoprotein by an ABC transporter modulator has been shown to alter the magnitude of volume-activated chloride currents (reviewed in Higgins, C. F. Volume-activated chloride currents associated with the multidrug resistance P-glycoprotein, J. Physiol. 482:31S-36S (1995)). Thus, in this model, p-glycoprotein and other ABC transporters have multiple functions, one of which is to allosterically modify the function of the other membrane proteins.

The present invention is consistent with a model in which the allosteric modification of other membrane proteins by e.g., an ABCG4 transporter, is responsible for a change in the transport of a substrate, e.g., β-amyloid, a cytotoxic drug, or other small molecule. Accordingly, an ABCG4 activity is at least one or more of the following activities:

1. activation of an ABCG4-dependent signal transduction pathway;

2. modulation of the transport of a substrate (e.g., a cytotoxic drug, β-amyloid) across a membrane;

3. interaction of an ABCG4 protein with a non-ABCG4 membrane-associated molecule;

4. modulation of the development or differentiation of an ABCG4-expressing cell;

5. modulation of the development or differentiation of a non-ABCG4-expressing cell;

6. modulation of the homeostasis of an ABCG4-expressing cell; and

7. modulation of the homeostasis of a non-ABCG4-expressing cell.

As used herein, an “ABCG4 transporter” (also referred to as “ABCG4 protein” or “ABCG4 polypeptide”) is a polypeptide having at least 50%, 60%, 70%, 75%, 80%, 85%, 90%, 95%, or 97% sequence identity with the full length sequence of human ABCG4 transporter (SEQ ID NOs: 2 or 13) and have at least one of the above functions. A “functional fragment” (also referred to as “biologically active portion”) refers to a portion of an ABCG4 transporter having at least one of the above functions.

Various aspects of the invention are described in further detail in the following subsections. The contents of all patents, patent applications, and references cited throughout the application are hereby incorporated by reference in their entireties.

A. Isolated Nucleic Acid Molecules

One aspect of the invention pertains to isolated nucleic acid molecules that encode ABCG4 transporter proteins or biologically active portions thereof, as well as nucleic acid fragments sufficient for use as hybridization probes to identify ABCG4-encoding nucleic acid molecules (e.g., ABCG4 transporter mRNA) and fragments for use as PCR primers for the amplification or mutation of ABCG4 transporter nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules that are separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated ABCG4 transporter nucleic acid molecule can contain less than about 5 kb, 4kb, 3kb, 2kb, 1 kb, 0.5 kb or 0.1 kb of nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NOs: 1, 3, or 12, or a portion thereof, can be isolated using standard molecular biology techniques and the sequence information provided herein. Using all or portion of the nucleic acid sequence of SEQ ID NOs: 1, 3, or 12 as a hybridization probe, ABCG4 transporter nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of SEQ ID NOs: 1, 3, or 12 can be isolated by the polymerase chain reaction (PCR) using synthetic oligonucleotide primers designed based upon the sequence of SEQ ID NOs: 1, 3, or 12.

A nucleic acid of the invention can be amplified using CDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to ABCG4 transporter nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NOs: 1, 3, or 12. The sequence of SEQ ID NOs: 1 or 12 corresponds to the human ABCG4 cDNAs. This cDNA comprises sequences encoding the human ABCG4 protein (i.e., “the coding region”, from nucleotides 7-1947), as well as 5′ untranslated sequences (nucleotides 1-6) and 3′ untranslated sequences (nucleotides 1948-3455). Alternatively, the nucleic acid molecule can comprise only the coding region of SEQ ID NO: 1 (e.g., nucleotides 7-1947, corresponding to SEQ ID NO: 3).

In another preferred embodiment, an isolated nucleic acid molecule of the invention comprises a nucleic acid molecule which is a complement of the nucleotide sequence shown in SEQ ID NOs: 1, 3, or 12, or a portion of any of these nucleotide sequences. A nucleic acid molecule which is complementary to the nucleotide sequence shown in SEQ ID NOs: 1, 3, or 12 is one which is sufficiently complementary to the nucleotide sequence shown in SEQ ID NOs: 1, 3, or 12 such that it can hybridize to the nucleotide sequence shown in SEQ ID NOs: 1, 3, or 12 thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 86%, 87%, 88%, 89%, 90%, 91%, 92%, 93%, 94%, 95%, 96%, 97%, 98%, or more identical to the entire length of the nucleotide sequence shown in SEQ ID NOs: 1, 3, or 12, or a portion of any of these nucleotide sequences. The identity algorithms and settings that may be used includes computer programs which employ the Smith-Waterman algorithm, such as the MPSRCH program (Oxford Molecular), using an affine gap search with the following parameters: a gap open penalty of 12 and a gap extension penalty of 1. Preferably, GCG PileUp program (Genetics Computer Group, Madison, Wis.) (Gapweight: 4, Gaplength weight: 1) is used for sequence alignment. Alternatively, the percent identity between two nucleotide sequences is determined using the GAβ program in the GCG software package (available at http://www.gcg.com), using a NWSgapdna.CMP matrix and a gap weight of 40, 50, 60, 70, or 80 and alength weight ofi, 2,3,4,5, or6.

Moreover, the nucleic acid molecule of the invention can comprise only a portion of the nucleic acid sequence of SEQ ID NOs: 1, 3, or 12, for example, a fragment which can be used as a probe or primer or a fragment encoding a portion of an ABCG4 transporter protein, e.g., a biologically active portion of an ABCG4 transporter protein. The nucleotide sequence determined from the cloning of the ABCG4 transporter gene allows for the generation of probes and primers designed for use in identifying and/or cloning other ABCG4 transporter family members, as well as ABCG4 transporter homologues from other species (also referred as “ABCG4 orthologues”). The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of an ABCG4 transporter gene (e.g., SEQ ID NOs: 1, 3, or 12), of an anti-sense sequence of an ABCG4 transporter gene, or of a naturally occurring allelic variant or mutant of a wild type ABCG4 gene. The term “wild type ABCG4 transporter gene,” as used herein, refers to an ABCG4 transporter gene present in the majority individual organisms of a selected population. In an exemplary embodiment, a nucleic acid molecule of the present invention comprises a nucleotide sequence which is greater than 50, 60, 70, 80, 90, 100, 150, 200, 300, 400, 500-1000, 1000-1500, 1500-2000, or 2000-2500 or more nucleotides in length and hybridizes under stringent hybridization conditions to a nucleic acid molecule of SEQ ID NOs: 1, 3, or 12. In another exemplary embodiment, a nucleic acid molecule of the present invention comprises at least 12 contiguous nucleotides of SEQ ID NOs: 1, 3, or 12, or a complement thereof from a region specific to ABCG4 transporters. A “region specific to ABCG4 transporters,” as used herein, includes 5′- and 3′-untranslated regions of an ABCG4 transporter gene, and coding regions encoding ABCG4-specific amino acid sequences that have less than 50%, preferably 40%, 30%, 20%, 10%, or 5% sequence homology with any region of the other ABC transporters. In a preferred embodiment, an oligonucleotide primer comprises the first 7 nucleotides (i.e., nucleotides 1-7) of SEQ ID NO: 1. In preferred embodiments, an oligonucleotide primer comprises the first 8, 9, 10, 11, 12, 13, 14 or 15 nucleotides of SEQ ID NO: 1.

Probes based on the ABCG4 transporter nucleotide sequences can be used to detect transcripts or genomic sequences encoding the same or homologous proteins. In preferred embodiments, the probe fuirther comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an ABCG4 transporter protein, such as by measuring a level of an ABCG4-encoding nucleic acid in a sample of cells from a subject e.g., detecting ABCG4 transporter mRNA levels or determining whether a genomic ABCG4 transporter gene has been mutated or deleted.

A nucleic acid fragment encoding a “biologically active portion of an ABCG4 transporter protein” can be prepared by isolating a portion of the nucleotide sequence of SEQ ID NOs: 1, 3, or 12, which encodes a polypeptide having an ABCG4 transporter biological activity (the biological activities of the ABCG4 transporter proteins are described herein), expressing the encoded portion of the ABCG4 transporter protein (e.g., by recombinant expression in vitro) and assessing the activity of the encoded portion of the ABCG4 transporter protein.

The invention further encompasses nucleic acid molecules that differ from the nucleotide sequence shown in SEQ ID NOs: 1, 3, or 12, due to degeneracy of the genetic code and thus encode the same ABCG4 transporter protein (i.e., SEQ ID NOs: 2 or 13) as those encoded by the nucleotide sequence shown in SEQ ID NOs: 1, 3, or 12.

In addition to the ABCG4 transporter nucleotide sequences shown in SEQ ID NOs: 1, 3, or 12, it will be appreciated by those skilled in the art that DNA sequence polymorphisms that lead to changes in the amino acid sequences of the ABCG4 transporter proteins may exist within a population (e.g., the human population). Such genetic polymorphism in the ABCG4 transporter genes may exist among individuals within a population due to natural allelic variation. As used herein, the term “ABCG4 gene” refers to a nucleic acid molecule which includes an open reading frame encoding an ABCG4 transporter protein, preferably a mammalian ABCG4 transporter protein, and can further include non-coding regulatory sequences, and introns.

The present invention also provides nucleic acids encoding a polypeptide comprising a naturally occurring allelic variant of an ABCG4 transporter. As used herein, a “naturally occurring allelic variant of an ABCG4 transporter” refers to a polypeptide that is encoded by a nucleic acid having the same chromosomal location as a wild type ABCG4 transporter gene, exists in nature, and is sufficiently homologous with a wild type ABCG4 transporter protein. Typically, an allelic variant of an ABCG4 transporter binds to an antibody that selectively binds to the polypeptide of SEQ ID NOs: 2 or 13.

Allelic variants of a human ABCG4 transporter include both functional and non-functional ABCG4 transporter proteins. Functional allelic variants are naturally occurring amino acid sequence variants of the human ABCG4 transporter that maintain the ability to bind an ABCG4 transporter ligand. Functional allelic variants will typically contain only conservative substitution of one or more amino acids of SEQ ID NOs: 2 or 13, or substitution, deletion or insertion of non-critical residues in non-critical regions of the protein.

Non-functional allelic variants are naturally occurring amino acid sequence variants of the human ABCG4 transporter protein that do not have the ability to either bind an ABCG4 transporter ligand. Non-functional allelic variants will typically contain a non-conservative substitution, a deletion, or insertion or premature truncation of the amino acid sequence of SEQ ID NOs: 2 or 13, or a substitution, insertion or deletion in critical residues or critical regions.

The present invention further provides nucleic acid sequences encoding non-human orthologues of the human ABCG4 transporter protein. Orthologues of the human ABCG4 transporter protein are proteins that are isolated from non-human organisms, have sequences more homologous to human ABCG4 transporter protein than to the other human ABC transporter proteins, and possess functions similar to those of the human ABCG4 transporter protein.

Nucleic acid molecules corresponding to natural allelic variants and homologues of the ABCG4 transporter cDNAs (including orthologues of human ABCG4 transporter gene) of the invention can be isolated based on their homology to the ABCG4 transporter nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the ABCG4 transporter cDNAs of the invention can further be isolated by mapping to the same chromosome or locus as the ABCG4 transporter gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NOs: 1, 3, or 12. In other embodiment, the nucleic acid is at least 30, 50, 100, 150, 200, 300, 400, 500, 600, 700, 800, 900, 1000, 1500, 2000, 2500, 3000, 3500, 4000, 4500, 5000, 5447 or more nucleotides in length. As used herein, the term “hybridizes under stringent conditions” is intended to describe conditions for hybridization and washing under which nucleotide sequences at least 75% homologous to each other typically remain hybridized to each other. Preferably, the conditions are such that sequences at least about 80%, even more preferably at least about 85%, 86%, 87%, 88%, 89%, 90%, 91%, 92%, 93%, 94%, 95%, 96%, 97%, or 98% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6X sodium chloride/sodium citrate (SSC) at about 45° C., followed by one or more washes in 0.2×SSC, 0.1% SDS at 50° C., preferably at 55° C., more preferably at 60° C., and even more preferably at 65° C. Preferably, an isolated nucleic acid molecule of the invention that hybridizes under stringent conditions to the sequence of SEQ ID NOs: 1, 3, or 12 corresponds to a naturally-occurring nucleic acid molecule.

In addition to naturally-occurring allelic variants of the ABCG4 transporter sequences that may exist in the population, the skilled artisan will further appreciate that changes can be introduced by mutation into the nucleotide sequences of SEQ ID NOs: 1, 3, or 12, thereby leading to changes in the amino acid sequence of the encoded ABCG4 transporter proteins, without altering the functional ability of the ABCG4 transporter proteins. For example, nucleotide substitutions leading to amino acid substitutions at “non-essential” amino acid residues can be made in the sequence of SEQ ID NOs: 1, 3, or 12. A “non-essential” amino acid residue is a residue that can be altered from the wild-type sequence of ABCG4 transporter (e.g., the sequence of SEQ ID NOs: 2 or 13) without altering the biological activity, whereas an “essential” amino acid residue is required for biological activity.

Accordingly, another aspect of the invention pertains to nucleic acid molecules encoding ABCG4 transporter proteins that contain changes in amino acid residues that are not essential for activity. Such ABCG4 transporter proteins differ in amino acid sequence from SEQ ID NOs: 2 or 13, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, or more homologous to SEQ ID NOs: 2 or 13.

An isolated nucleic acid molecule encoding an ABCG4 transporter protein homologous to the protein of SEQ ID NOs: 2 or 13 can be created by introducing one or more nucleotide substitutions, additions, or deletions into the nucleotide sequence of SEQ ID NOs: 1, 3, or 12, such that one or more amino acid substitutions, additions or deletions are introduced into the encoded protein. Mutations can be introduced into SEQ ID NOs: 1, 3, or 12 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an ABCG4 transporter protein is preferably replaced with another amino acid residue from the same side chain family. Alternatively, in another embodiment, mutations can be introduced randomly along all or part of an ABCG4 transporter coding sequence, such as by saturation mutagenesis, and the resultant mutants can be screened for ABCG4 transporter biological activity to identify mutants that retain activity. Following mutagenesis of SEQ ID NOs: 1, 3, or 12, the encoded protein can be expressed recombinantly and the activity of the protein can be determined.

In a preferred embodiment, a mutant ABCG4 transporter protein can be assayed for the ability to interact with a non-ABCG4 transporter molecule, e.g., an ABCG4 transporter ligand, e.g., a polypeptide or a small molecule.

In addition to the nucleic acid molecules encoding ABCG4 transporter proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire ABCG4 transporter coding strand, or to only a portion thereof. In one embodiment, an antisense nucleic acid molecule is antisense to a “coding region” of the coding strand of a nucleotide sequence encoding ABCG4. The term “coding region” refers to the region of the nucleotide sequence comprising codons which are translated into amino acid residues (e.g., the coding region of human ABCG4 corresponds to SEQ ID NO: 3). In another embodiment, the antisense nucleic acid molecule is antisense to a “noncoding region” of the coding strand of a nucleotide sequence encoding ABCG4. The term “noncoding region” refers to 5′ and 3′ sequences which flank the coding region that are not translated into amino acids (i.e., also referred to as 5′ and 3′ untranslated regions).

Given the coding strand sequences encoding ABCG4 transporter disclosed herein (e.g., SEQ ID NO: 3), antisense nucleic acids of the invention can be designed according to the rules of Watson and Crick base pairing. The antisense nucleic acid molecule can be complementary to the entire coding region of ABCG4 transporter mRNA, but more preferably is an oligonucleotide which is antisense to only a portion of the coding or noncoding region of ABCG4 transporter MRNA. For example, the antisense oligonucleotide can be complementary to the region surrounding the translation start site of ABCG4 transporter mRNA. An antisense oligonucleotide can be, for example, about 5, 10, 15, 20, 25, 30, 35, 40, 45 or 50 nucleotides or more in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an ABCG4 transporter protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an MRNA, to which they have a complementary region. Thus, ribozymes (e.g., hammerhead ribozymes (described in Haselhoff and Gerlach (1988) Nature 334:585-591)) can be used to catalytically cleave ABCG4 transporter mRNA transcripts to thereby inhibit translation of ABCG4 transporter mRNA. A ribozyme having specificity for an ABCG4-encoding nucleic acid can be designed based upon the nucleotide sequence of an ABCG4 transporter cDNA disclosed herein (i.e., SEQ ID NO: 1). For example, a derivative of a Tetrahymena L-19 IVS RNA can be constructed in which the nucleotide sequence of the active site is complementary to the nucleotide sequence to be cleaved in an ABCG4-encoding mRNA. See, e.g., Cech et al. U.S. Pat. No. 4,987,071; and Cech et al. U.S. Pat. No. 5,116,742. Alternatively, ABCG4 transporter mRNA can be used to select a catalytic RNA having a specific ribonuclease activity from a pool of RNA molecules. See, e.g., Bartel, D. and Szostak, J. W. (1993) Science 261:1411-1418.

Alternatively, ABCG4 transporter gene expression can be inhibited by targeting nucleotide sequences complementary to the regulatory region of the ABCG4 transporter (e.g., the ABCG4 transporter promoter and/or enhancers) to form triple helical structures that prevent transcription of the ABCG4 transporter gene in target cells. See generally, Helene, C. (1991) Anticancer Drug Des. 6(6):569-84; Helene, C. et al. (1992) Ann. N.Y. Acad. Sci. 660:27-36; and Maher, L. J. (I1992) Bioassays 14(12):807-15.

In yet another embodiment, the ABCO4 transporter nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (I1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of ABCG4 transporter nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of ABCG4 transporter nucleic acid molecules can also be used in the analysis of single base pair mutations in a gene, (e.g., by PNA-directed PCR clamping); as ‘artificial restriction enzymes’ when used in combination with other enzymes, (e.g, S1 nucleases (Hyrup B. (1996) supra)); or as probes or primers for DNA sequencing or hybridization (Hyrup B. et al. (1996) supra; Perry-O'Keefe supra).

In another embodiment, PNAs of ABCG4 transporter nucleic acid molecules can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of ABCG4 transporter nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al (1989) Proc. Natl. Acad. Sci. USA 86:6553-6556; Lemaitre et al (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

B. Isolated ABCG4 transporter proteins and Anti-ABCG4 transporter Antibodies

One aspect of the invention pertains to isolated ABCG4 transporter proteins, and biologically active portions thereof, as well as polypeptide fragments suitable for use as immunogens to raise anti-ABCG4 transporter antibodies. In one embodiment, the isolated polypeptide comprises the amino acid sequence of SEQ ID NOs: 2 or 13, or a functional fragment thereof. In another embodiment, the isolated polypeptide comprises a naturally occurring allelic variant of the amino acid sequence of SEQ ID NOs: 2 or 13. Typically, the allelic variant binds to an antibody that selectively binds to the polypeptide of SEQ ID NOs: 2 or 13. In another embodiment, the isolated polypeptide comprises a functional fragment of the naturally occurring allelic variant described above. In another embodiment, the isolated polypeptide comprises an amino acid sequence which is at least about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or more similar to the amino acid sequence of SEQ ID NOs: 2 or 13. In yet another embodiment, the isolated polypeptide comprises a functional fragment of the polypeptide having a sequence similar to the amino acid sequence of SEQ ID NOs: 2 or 13 as described above.

Native ABCG4 transporter proteins or functional fragments thereof of the present invention can be isolated from cells or tissue sources by an appropriate purification scheme using standard protein purification techniques. Alternatively, ABCG4 transporter proteins or functional fragments thereof are produced by recombinant DNA techniques. In addition, an ABCG4 transporter protein or polypeptide can be synthesized chemically using standard peptide synthesis techniques.

An “isolated” or “purified” protein or biologically active portion thereof is substantially free of cellular material or other contaminating proteins from the cell or tissue source from which the ABCG4 transporter protein is derived, or substantially free from chemical precursors or other chemicals when chemically synthesized. The language “substantially free of cellular material” includes preparations of ABCG4 transporter protein in which the protein is separated from cellular components of the cells from which it is isolated or recombinantly produced. In one embodiment, the language “substantially free of cellular material” includes preparations of ABCG4 transporter protein having less than about 30% (by dry weight) of non-ABCG4 transporter protein (also referred to herein as a “contaminating protein”), more preferably less than about 20% of non-ABCG4 transporter protein, still more preferably less than about 10% of non-ABCG4 transporter protein, and most preferably less than about 5% non-ABCG4 transporter protein. When the ABCG4 transporter protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of ABCG4 transporter protein in which the protein is separated from chemical precursors or other chemicals which are involved in the synthesis of the protein. In one embodiment, the language “substantially free of chemical precursors or other chemicals” includes preparations of ABCG4 transporter protein having less than about 30% (by dry weight) of chemical precursors or non-ABCG4 transporter chemicals, more preferably less than about 20% chemical precursors or non-ABCG4 transporter chemicals, still more preferably less than about 10% chemical precursors or non-ABCG4 transporter chemicals, and most preferably less than about 5% chemical precursors or non-ABCG4 transporter chemicals.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 70%, preferably, 80%, 90% or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

A comparison of sequences and determination of percent identity and/or similarity between two sequences can be accomplished using a mathematical algorithm. In a preferred embodiment, the percent identity between two amino acid sequences is determined using standard art recognized comparison software using standard parameter settings. For example, the Needleman and Wunsch (J. Mol. Biol. (48):444-453 (1970)) algorithm which has been incorporated into the GAβ program in the GCG software package (available at http://www.gcg.com) can be employed using either a Blossum 62 matrix or a PAM250 matrix, and a gap weight of 16, 14, 12, 10, 8, 6, or 4 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to ABCG4 transporter nucleic acid molecules of the invention. BLAST protein searches can be performed with the XBLAST program, score=50, wordlength=3 to obtain amino acid sequences homologous to ABCG4 transporter protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

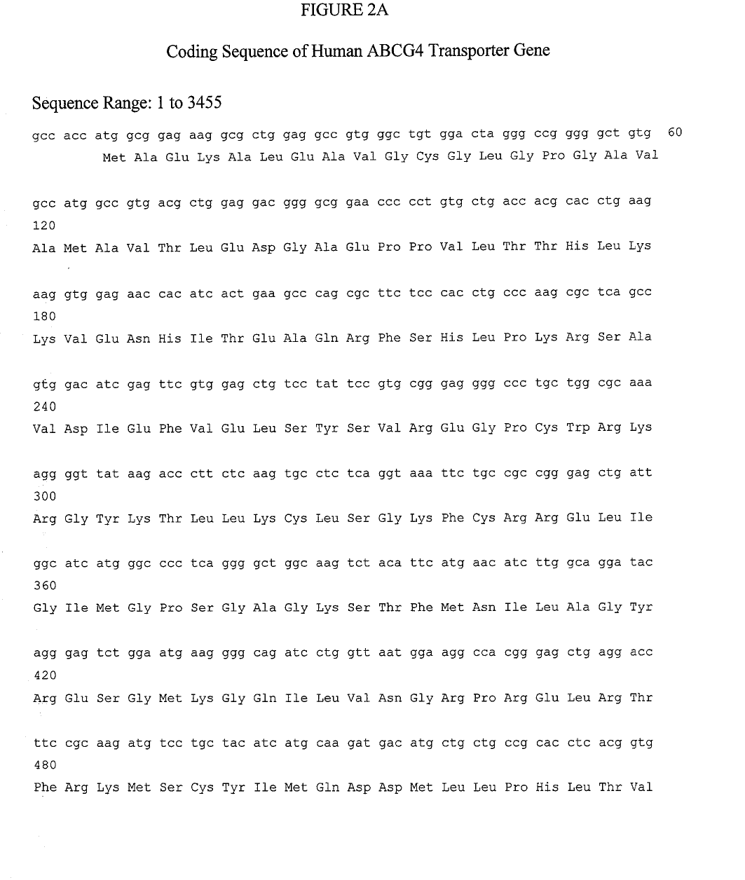

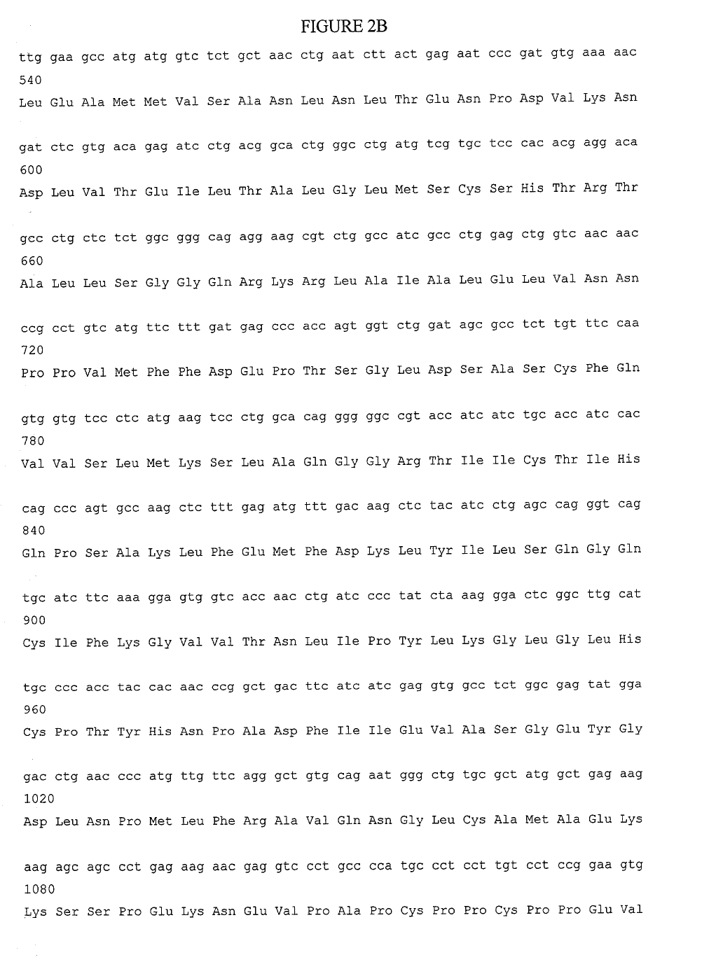

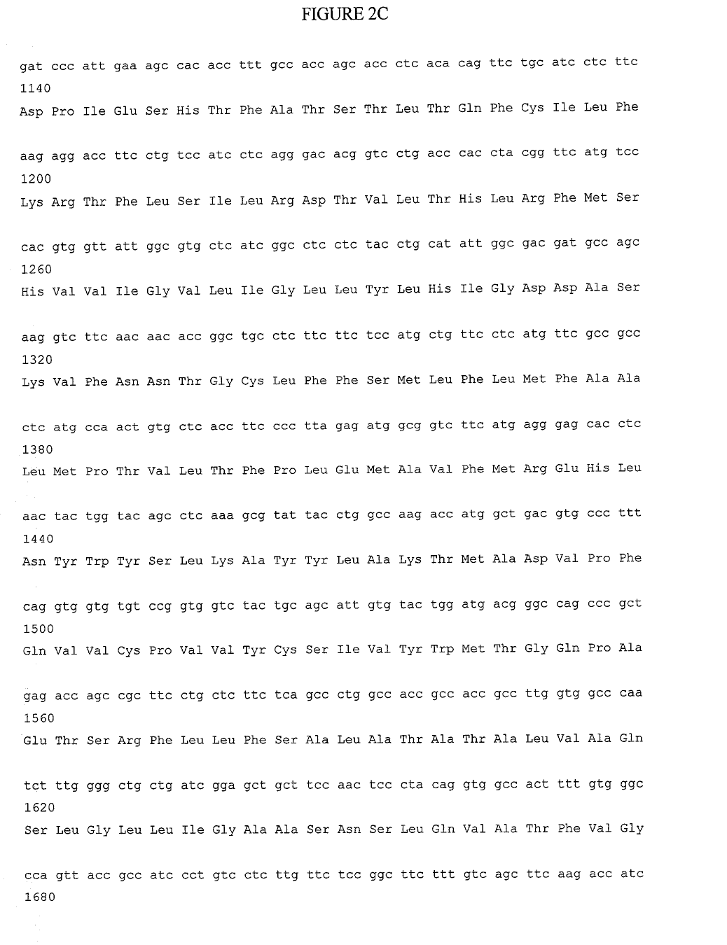

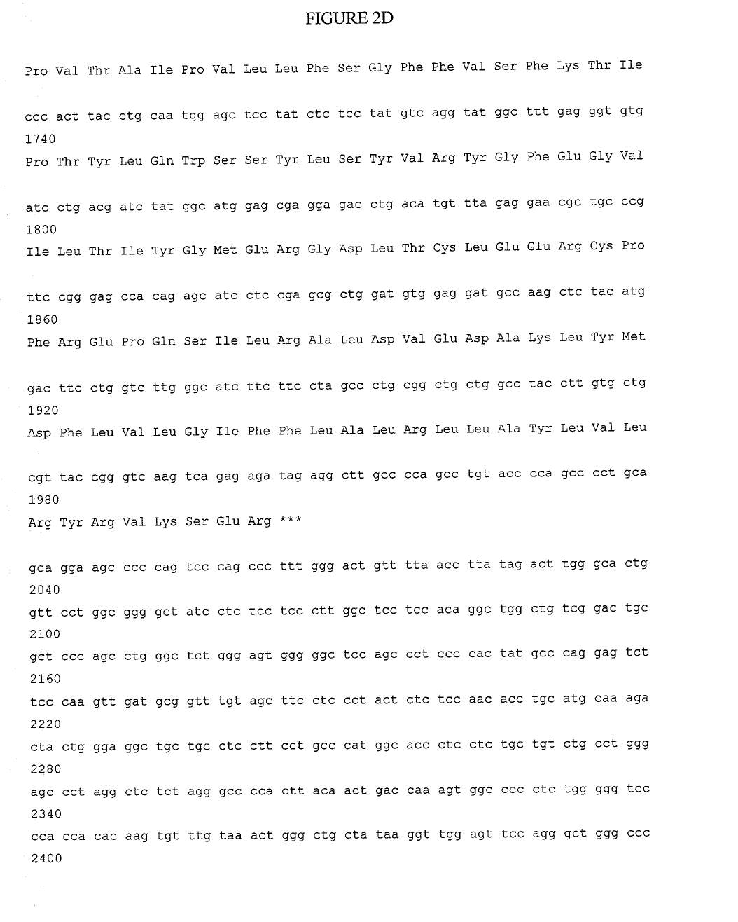

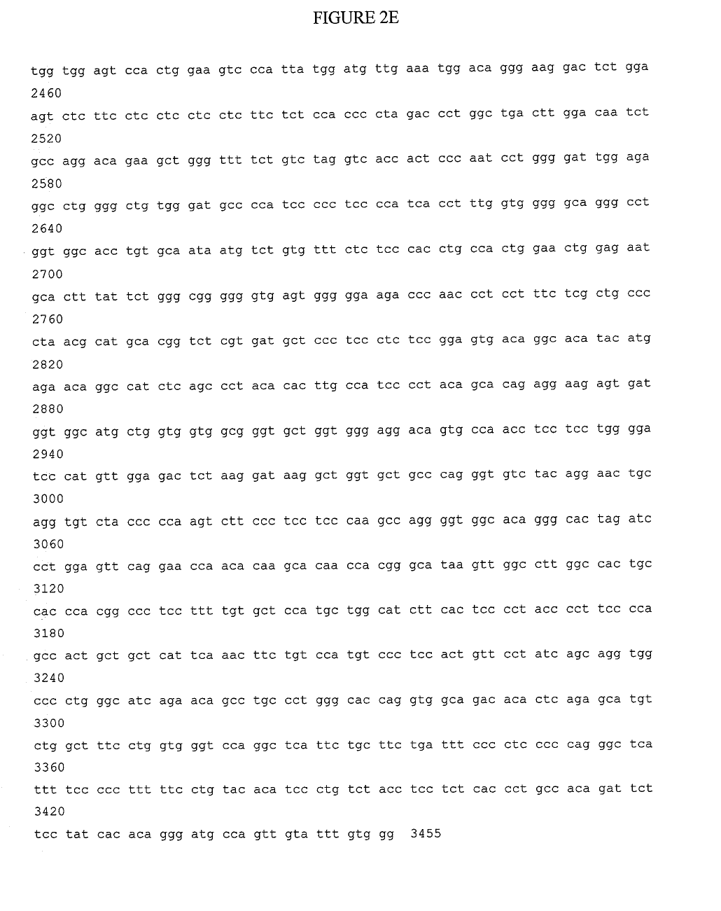

The present invention provides polypeptides comprising a functional fragment (also referred to as “a biologically active portion”) of an ABCG4 transporter protein. Typically, biologically active portions comprise a domain or motif with at least one activity of the ABCG4 transporter protein. A biologically active portion of an ABCG4 transporter protein can be a polypeptide which is, for example, 10, 25, 50, 100, 200, 300, 400, 500, 600, 700, 800 or more amino acids in length. In one embodiment, a biologically active portion of an ABCG4 transporter protein comprises at least one transmembrane domain. In another embodiment, biologically active portion of an ABCG4 transporter protein contains at least two transmembrane domains. One or more of these transmembrane domains may associate to form a membrane-spanning domain. In addition, or alternatively, the biologically active portion of the ABCG4 transporter protein may include multiple clusters of conserved residues that define an ATP binding domain. In addition, or alternatively, the biologically active portion of the ABCG4 transporter protein may comprise a Walker domain, e.g., a Walker A and/or Walker B domain (see FIG. 2.; Patel et al. (1998) Trends Cell Biol 8: 65-71). In a preferred embodiment, the biological active portion of the ABCG4 transporter protein participates in an interaction between an ABCG4 transporter molecule and a non-ABCG4 transporter molecule. Identification of these domains may be facilitated using any of a number of art recognized molecular modeling techniques as described herein. Moreover, other biologically active portions, in which other regions of the protein are deleted, can be prepared by recombinant techniques and evaluated for one or more of the functional activities of a native ABCG4 transporter protein. The identified biologically active portions can be used as targets for developing agents which modulate an ABCG4 transporter mediated activity.

The invention also provides ABCG4 transporter chimeric or fusion proteins. As used herein, an ABCG4 transporter “chimeric protein” or “fusion protein” comprises an ABCG4 transporter polypeptide or a functional fragment thereof operatively linked to a heterologous polypeptide. A “heterologous polypeptide” refers to a polypeptide that is not a portion of the full length ABCG4 transporter at least a portion of which is fused to the heterologous polypeptide. Thus, the fusion protein of the present invention may comprise a portion of an ABCG4 transporter (e.g., a human ABCG4 transporter) and a portion of another ABCG4 transporter (e.g., a mouse ABCG4 transporter). In a preferred embodiment, an ABCG4 transporter fusion protein comprises at least one biologically active portion of an ABCG4 transporter protein. In another preferred embodiment, an ABCG4 transporter fusion protein comprises at least two biologically active portions of an ABCG4 transporter protein. In yet another preferred embodiment, an ABCG4 transporter fusion protein comprises a non-ABCG4 transporter polypeptide. A “non-ABCG4 transporter polypeptide,” as used herein, refers to a polypeptide having an amino acid sequence having an amino acid sequence corresponding to a protein which is not sufficiently homologous to the ABCG4 transporter protein. Within the fusion protein, the term “operatively linked” is intended to indicate that the ABCG4 transporter polypeptide and the non-ABCG4 transporter polypeptide are fused in-frame to each other. The heterologous polypeptide can be fused to the N-terminus or C-terminus of an ABCG4 transporter polypeptide or a functional fragment thereof.

In one embodiment, the fusion protein is a GST-ABCG4 transporter fusion protein in which the ABCG4 transporter sequences are fused to the C-terminus of the GST sequences. Such fusion proteins can facilitate the purification of recombinant ABCG4.

In another embodiment, the fusion protein is an ABCG4 transporter protein containing a heterologous signal sequence at its N-terminus. In certain host cells (e.g., mammalian host cells), expression and/or secretion of ABCG4 transporter can be increased through use of a heterologous signal sequence.

The ABCG4 transporter fusion proteins of the invention can be incorporated into pharmaceutical compositions and administered to a subject in vivo. The ABCG4 transporter fusion proteins can be used to affect the bioavailability of an ABCG4 transporter substrate. Use of ABCG4 transporter fusion proteins may be useful therapeutically for the treatment of disorders caused by, for example, (i) aberrant modification or mutation of a gene encoding an ABCG4 transporter protein; (ii) mis-regulation of the ABCG4 transporter gene; and (iii) aberrant post-translational modification of an ABCG4 transporter protein.

Moreover, the ABCG4-fusion proteins of the invention can be used as immunogens to produce anti-ABCG4 transporter antibodies in a subject, to purify ABCG4 transporter ligands and in screening assays to identify molecules which inhibit the interaction of an ABCG4 transporter with an ABCG4 transporter substrate.

Preferably, an ABCG4 transporter chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An ABCG4 transporter-encoding nucleic acid can be cloned into such an expression vector such that the fusion moiety is linked in-frame to the ABCG4 transporter protein.

The present invention also pertains to variants of the ABCG4 transporter proteins which function as either ABCG4 transporter agonists or as ABCG4 transporter antagonists. Variants of the ABCG4 transporter proteins can be generated by mutagenesis, e.g., discrete point mutation or truncation of an ABCG4 transporter protein. An agonist of the ABCG4 transporter proteins can retain substantially the same, or a subset, of the biological activities of the naturally occurring form of an ABCG4 transporter protein. An antagonist of an ABCG4 transporter protein can inhibit one or more of the activities of the naturally occurring form of the ABCG4 transporter protein by, for example, competitively modulating an activity of an ABCG4 transporter protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the ABCG4 transporter protein.

In one embodiment, variants of an ABCG4 transporter protein which function as either ABCG4 transporter agonists (mimetics) or as ABCG4 transporter antagonists can be identified by screening combinatorial libraries of mutants, e.g., truncation mutants, of an ABCG4 transporter protein for ABCG4 transporter protein agonist or antagonist activity. In one embodiment, a variegated library of ABCG4 transporter variants is generated by combinatorial mutagenesis at the nucleic acid level and is encoded by a variegated gene library. A variegated library of ABCG4 transporter variants can be produced by, for example, enzymatically ligating a mixture of synthetic oligonucleotides into gene sequences such that a degenerate set of potential ABCG4 transporter sequences is expressible as individual polypeptides, or alternatively, as a set of larger fusion proteins (e.g., for phage display) containing the set of ABCG4 transporter sequences therein. There are a variety of methods which can be used to produce libraries of potential ABCG4 transporter variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential ABCG4 transporter sequences. Methods for synthesizing degenerate oligonucleotides are known in the art (see, e.g., Narang, S.A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of an ABCG4 transporter protein coding sequence can be used to generate a variegated population of ABCG4 transporter fragments for screening and subsequent selection of variants of an ABCG4 transporter protein. In one embodiment, a library of coding sequence fragments can be generated by treating a double stranded PCR fragment of an ABCG4 transporter coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with Sl nuclease, and ligating the resulting fragment library into an expression vector. By this method, an expression library can be derived which encodes N-terminal, C-terminal and internal fragments of various sizes of the ABCG4 transporter protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of ABCG4 transporter proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify ABCG4 transporter variants (Arkin and Yourvan (1992) Proc. Natl. Acad. Sci. USA 89:7811-7815; Delgrave et al. (1993) Protein Engineering 6(3):327-33 1).

An isolated ABCG4 transporter protein, or a portion or fragment thereof, can be used as an immunogen to generate antibodies that bind ABCG4 transporter using standard techniques for polyclonal and monoclonal antibody preparation. A full-length ABCG4 transporter protein can be used or, alternatively, the invention provides antigenic peptide fragments of ABCG4 transporter for use as immunogens. Preferred epitopes encompassed by the antigenic peptide are regions of ABCG4 transporter that are located on the surface of the protein, e.g., hydrophilic regions, as well as regions with high antigenicity. Other preferred epitopes are ABCG4 transporter-specific regions.

An ABCG4 transporter immunogen typically is used to prepare antibodies by immunizing a suitable subject, (e.g., rabbit, goat, mouse or other mammal) with the immunogen. An appropriate immunogenic preparation can contain, for example, recombinantly expressed ABCG4 transporter protein or a chemically synthesized ABCG4 transporter polypeptide. The preparation can further include an adjuvant, such as Freund's complete or incomplete adjuvant, or similar immunostimulatory agent. Immunization of a suitable subject with an immunogenic ABCG4 transporter preparation induces a polyclonal anti-ABCG4 transporter antibody response.

Accordingly, another aspect of the invention pertains to anti-ABCG4 transporter antibodies. The term “antibody” as used herein refers to immunoglobulin molecules and immunologically active portions of immunoglobulin molecules, i.e., molecules that contain an antigen binding site which specifically binds (immunoreacts with) an antigen, such as ABCG4 transporter. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind ABCG4 transporter. The term “monoclonal antibody” or “monoclonal antibody composition”, as used herein, refers to a population of antibody molecules that contain only one species of an antigen binding site capable of immunoreacting with a particular epitope of ABCG4 transporter. A monoclonal antibody composition thus typically displays a single binding affinity for a particular ABCG4 transporter protein with which it immunoreacts.

Polyclonal anti-ABCG4 transporter antibodies can be prepared as described above by immunizing a suitable subject with an ABCG4 transporter immunogen. The anti-ABCG4 transporter antibody titer in the immunized subject can be monitored over time by standard techniques, such as with an enzyme linked immunosorbent assay (ELISA) using immobilized ABCG4 transporter. If desired, the antibody molecules directed against ABCG4 transporter can be isolated from the mammal (e.g., from the blood) and firther purified by well known techniques, such as protein A chromatography to obtain the IgG fraction. At an appropriate time after immunization, e.g., when the anti-ABCG4 transporter antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al (1981) J. Immunol. 127:539-46; Brown et al. (1980) J. Biol. Chem. 255:4980-83; Yeh et al. (1976) Proc. Natl. Acad. Sci. USA 76:2927-3 1; and Yeh et al. (1982) Int. J Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, New York (1980); E. A. Lemer (1981) Yale J Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an ABCG4 transporter immunogen as described above, and the culture supernatants of the resulting hybridoma cells are screened to identify a hybridoma producing a monoclonal antibody that binds ABCG4 transporter.

Any of the many well known protocols used for fusing lymphocytes and immortalized cell lines can be applied for the purpose of generating an anti-ABCG4 transporter monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1I/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind ABCG4, e.g., using a standard ELISA assay.

Alternative to preparing monoclonal antibody-secreting hybridomas, a monoclonal anti-ABCG4 transporter antibody can be identified and isolated by screening a recombinant combinatorial immunoglobulin library (e.g., an antibody phage display library) with ABCG4 transporter to thereby isolate immunoglobulin library members that bind ABCG4. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-ABCG4 transporter antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-ABCG4 transporter antibody (e.g., monoclonal antibody) can be used to isolate ABCG4 transporter by standard techniques, such as affinity chromatography or immunoprecipitation. An anti-ABCG4 transporter antibody can facilitate the purification of natural ABCG4 transporter from cells and of recombinantly produced ABCG4 transporter expressed in host cells. Moreover, an anti-ABCG4 transporter antibody can be used to detect ABCG4 transporter protein (e.g., in a cellular lysate or cell supernatant) in order to evaluate the abundance and pattern of expression of the ABCG4 transporter protein. Anti-ABCG4 transporter antibodies can be used diagnostically to monitor protein levels in tissue as part of a clinical testing procedure, e.g., to, for example, determine the efficacy of a given treatment regimen. Detection can be facilitated by coupling (ie., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³³P, ³²P, or ³H.

C. Recombinant Expression Vectors and Host Cells

Another aspect of the invention pertains to vectors, preferably expression vectors, containing a nucleic acid encoding an ABCG4 transporter protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fiusion proteins or peptides, encoded by nucleic acids as described herein (e.g., ABCG4 transporter proteins, mutant forms of ABCG4 transporter proteins, fusion proteins, and the like).

The recombinant expression vectors of the invention can be designed for expression of ABCG4 transporter proteins in prokaryotic or eukaryotic cells. For example, ABCG4 transporter proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fiusion or non-fision proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in ABCG4 transporter activity assays, (e.g., direct assays or competitive assays described in detail below), or to generate antibodies specific for ABCG4 transporter proteins, for example. In a preferred embodiment, an ABCG4 transporter fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 1 ld (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gnl). This viral polymerase is supplied by host strains BL21(DE3) or HMS174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the ABCG4 transporter expression vector is a yeast expression vector. Examples of vectors for expression in yeast S. cerivisae include pYepSecl (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, ABCG4 transporter proteins can be expressed in insect cells using baculovirus expression vectors. Baculovirus vectors available for expression of proteins in cultured insect cells (e.g., Sf 9 cells) include the pAc series (Smith et al. (1983) Mol Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control finctions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Baneiji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to ABCG4 transporter mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which an ABCG4 transporter nucleic acid molecule of the invention is introduced, e.g., an ABCG4 transporter nucleic acid molecule within a recombinant expression vector or an ABCG4 transporter nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an ABCG4 transporter protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an ABCG4 transporter protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an ABCG4 transporter protein. Accordingly, the invention further provides methods for producing an ABCG4 transporter protein using the host cells of the invention. In one embodiment, the method comprises culturing the host cell of the invention (into which a recombinant expression vector encoding an ABCG4 transporter protein has been introduced) in a suitable medium such that an ABCG4 transporter protein is produced. In another embodiment, the method further comprises isolating an ABCG4 transporter protein from the medium or the host cell.

The host cells of the invention can also be used to produce non-human transgenic animals. For example, in one embodiment, a host cell of the invention is a fertilized oocyte or an embryonic stem cell into which ABCG4-coding sequences have been introduced. Such host cells can then be used to create non-human transgenic animals in which exogenous ABCG4 transporter sequences have been introduced into their genome or homologous recombinant animals in which endogenous ABCG4 transporter sequences have been altered. Such animals are useful for studying the function and/or activity of an ABCG4 transporter and for identifying and/or evaluating modulators of ABCG4 transporter activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous ABCG4 transporter gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an ABCG4-encoding nucleic acid into the male pronuclei of a fertilized oocyte, e.g., by microinjection, retroviral infection, and allowing the oocyte to develop in a pseudopregnant female foster animal. The ABCG4 transporter cDNA sequence of SEQ ID NO: 1 can be introduced as a transgene into the genome of a non-human animal. Alternatively, a nonhuman homologue of a human ABCG4 transporter gene, such as a mouse or rat ABCG4 transporter gene, can be used as a transgene. Alternatively, an ABCG4 transporter gene homologue, such as another ABC transporter family member, can be isolated based on hybridization to the ABCG4 transporter cDNA sequences of SEQ ID NOs: 1, 3, or 12 (described further in subsection I above) and used as a transgene. Intronic sequences and polyadenylation signals can also be included in the transgene to increase the efficiency of expression of the transgene. A tissue-specific regulatory sequence(s) can be operably linked to an ABCG4 transporter transgene to direct expression of an ABCG4 transporter protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an ABCG4 transporter transgene in its genome and/or expression of ABCG4 transporter mRNA in tissues or cells of the animals. A transgenic founder animal can then be used to breed additional animals carrying the transgene. Moreover, transgenic animals carrying a transgene encoding an ABCG4 transporter protein can further be bred to other transgenic animals carrying other transgenes, for example, animals carrying a transgene encoding a neurotoxic polypeptide such as β-amyloid.

To create a homologous recombinant animal, a vector is prepared which contains at least a portion of an ABCG4 transporter gene into which a deletion, addition or substitution has been introduced to thereby alter, e.g., functionally disrupt, the ABCG4 transporter gene. The ABCG4 transporter gene can be a human gene (e.g., the cDNA of SEQ ID NO: 3), but more preferably, is a non-human homologue of a human ABCG4 transporter gene (e.g., a cDNA isolated by stringent hybridization with the nucleotide sequence of SEQ ID NOs: 1 or 12). For example, a mouse ABCG4 transporter gene can be used to construct a homologous recombination nucleic acid molecule, e.g., a vector, suitable for altering an endogenous ABCG4 transporter gene in the mouse genome. In a preferred embodiment, the homologous recombination nucleic acid molecule is designed such that, upon homologous recombination, the endogenous ABCG4 transporter gene is functionally disrupted (i.e., no longer encodes a functional protein; also referred to as a “knock out” vector). Alternatively, the homologous recombination nucleic acid molecule can be designed such that, upon homologous recombination, the endogenous ABCG4 transporter gene is mutated or otherwise altered but still encodes functional protein (e.g., the upstream regulatory region can be altered to thereby alter the expression of the endogenous ABCG4 transporter protein). In the homologous recombination nucleic acid molecule, the altered portion of the ABCG4 transporter gene is flanked at its 5′ and 3′ ends by additional nucleic acid sequence of the ABCG4 transporter gene to allow for homologous recombination to occur between the exogenous ABCG4 transporter gene carried by the homologous recombination nucleic acid molecule and an endogenous ABCG4 transporter gene in a cell, e.g., an embryonic stem cell. The additional flanking ABCG4 transporter nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced ABCG4 transporter gene has homologously recombined with the endogenous ABCG4 transporter gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The DNA sequences described herein can also be used to introduce a desired ABC transporter nucleotide sequence within a predicted location of the targeted genome, leading to the replacement of a copy of the targeted gene by another copy sufficiently homologous to allow a homologous recombination event to occur (knock-in homologous recombination). A knock-in of a target gene means an alteration in a host cell genome that results in altered expression, such as increased expression of the target gene, for example by introduction of an additional copy of the target gene, or by operatively inserting a regulatory sequence that provides for enhanced expression of an endogenous copy of the target gene. See, for example, U.S. Pat. Nos. 6,175,057; 6,335,180; and 6,194,633.

D. Pharmaceutical Compositions

The ABCG4 transporter nucleic acid molecules, portions of ABCG4 transporter genes encoding function fragments of ABCG4, fragments of ABCG4 transporter proteins, anti-ABCG4 transporter antibodies (also referred to herein as “active compounds”), antisense oligonucleotides capable of specifically hybridizing to a portion, or the full length of, ABCG4 genes, or any compound identified as a modulator of an ABCG4 transporter (as described herein) can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an ABCG4 transporter protein or an anti-ABCG4 transporter antibody) in the required amount in an appropriate solvent with one or a combination of ingredients enumerated above, as required, followed by filtered sterilization. Generally, dispersions are prepared by incorporating the active compound into a sterile vehicle which contains a basic ABCG4 transporter dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

E. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharnacogenetics); and c) methods of treatment (e.g., therapeutic and prophylactic).

The isolated nucleic acid molecules of the invention can be used, for example, to express ABCG4 transporter protein (e.g., via a recombinant expression vector in a host cell in gene therapy applications), to detect ABCG4 transporter mRNA (e.g., in a biological sample) or a genetic alteration in an ABCG4 transporter gene, and to modulate ABCG4 transporter activity, as described further below. The ABCG4 transporter proteins can be used to treat disorders characterized by insufficient or excessive production of an ABCG4 transporter substrate or production of ABCG4 transporter inhibitors. In addition, the ABCG4 transporter proteins can be used to screen for naturally occurring ABCG4 transporter substrates, to screen for drugs or compounds which modulate ABCG4 transporter activity, as well as to treat disorders characterized by insufficient or excessive production of ABCG4 transporter protein or production of ABCG4 transporter protein forms which have decreased, aberrant or unwanted activity compared to ABCG4 transporter wild type protein. Moreover, the anti-ABCG4 transporter antibodies of the invention can be used to detect and isolate ABCG4 transporter proteins, regulate the bioavailability of ABCG4 transporter proteins, and modulate ABCG4 transporter activity.

1. Screening Assays:

The invention provides a method (also referred to herein as a “screening assay”) for identifying modulators, i.e., candidate or test compounds or agents which bind to ABCG4 transporter proteins, have a stimulatory or inhibitory effect on, for example, ABCG4 transporter expression or ABCG4 transporter activity, or have a stimulatory or inhibitory effect on, for example, the expression or activity of an ABCG4 transporter substrate. Such modulaters may be peptides, peptidomimetics, small molecules or other drugs.

In one embodiment, the invention provides assays for screening candidate or test compounds which are substrates of an ABCG4 transporter protein or polypeptide or biologically active portion thereof. In another embodiment, the invention provides assays for screening candidate or test compounds which bind to or modulate the activity of an ABCG4 transporter protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K.S. (1997) Anticancer Drug Des. 12:145).

Examples of methods for the synthesis of molecular libraries can be found in the art, for example in: DeWitt et al. (1 993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner USP 5,223,409), spores (Ladner U.S. Pat. No. '409), plasmids (Cull et al. (1992) Proc Nat'l Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an ABCG4 transporter protein or biologically active portion thereof is contacted with a test compound and the ability of the test compound to modulate ABCG4 transporter activity is determined. Determining the ability of the test compound to modulate ABCG4 transporter activity can be accomplished by monitoring, for example, cellular transport of organic anions, organic cations, cytotoxic anti-cancer drugs, intracellular calcium, potassium, phosphatidylcholine, sodium concentration, neuronal membrane depolarization, a neurotoxic polypeptide (eg, β-amyloid), or the activity of an ABCG4 transporter-regulated transcription factor. The cell, for example, can be of mammalian origin, e.g., a neuronal cell. The ability of the test compound to modulate ABCG4 transporter binding to a substrate or to bind to ABCG4 transporter can also be determined. Determining the ability of the test compound to modulate ABCG4 transporter binding to a substrate can be accomplished, for example, by coupling the ABCG4 transporter substrate with a radioisotope or enzymatic label such that binding of the ABCG4 transporter substrate to ABCG4 transporter can be determined by detecting the labeled ABCG4 transporter substrate in a complex. Determining the ability of the test compound to bind ABCG4 transporter can be accomplished, for example, by coupling the compound with a radioisotope or enzymatic label such that binding of the compound to ABCG4 transporter can be determined by detecting the labeled ABCG4 transporter compound in a complex. For example, compounds (e.g., ABCG4 transporter substrates) can be labeled with ¹²⁵I, ³⁵S, ¹⁴C, ³³P, ³²P, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In one embodiment, suitable compounds include, but are not limited to, verapamil, desmethoxyverapamil, chloroquine, quinine, chinchonidine, primaquine, tamoxifen, dihydrocyclosporin, yohimbine, corynanthine, reserpine, physostigmine, acridine, acridine orange, quinacrine, trifluoroperazine chlorpromazine, propanolol, atropine, tryptamine, forskolin, 1,9-dideoxyforskolin, cyclosporin, (U.S. Pat. No. 4,117,118 (1978)), PSC-833 (cyclosporin D, 6-[(2S, 4R, 6E)-4-methyl-2-(methylamino)-3-oxo-6-octenoic acid]-(9CI)), [U.S. Pat. No. 5,525,590] [ACS 121584-18-7], Keller et al., “SDZ PSC 833, a non-immunosuppressive cylcosporine: its potency in overcoming p-glycoprotein-mediated multidrug resistance of murine leukemia”, Int J Cancer 50:593-597 (1992)), RU-486 (1703-hydroxy-11β-[4-dimethylaminophenyl]-17α prop-1-ynyl estrα-4, 9-dien-3 one), RU-49953 (17β-hydroxy-11β, 17α-[4-dimethylaminophenyl]-17α prop-1-ynyl estra-4, 9 dien-3 one), S9778 (6-{4-[2,2-di( )-ethylamino]-1-piperidinyl}-N,N′, di-2-propenyl-1,3,5-triazine-2,4-diamine, bismethane sulfonate, [U.S. Pat. No. 5,225,411; EP 466586] [ACS # 140945-01-3]; Dhainaut et al., “New triazine derivatives as potent modulators of multidrug resistance,” J Medicinal Chemistry 35:2481-2496 (1992)), MS-209 (5-[3-[4-(2,2-diphenylacetyl)piperazin-1-yl]-2-hydroxypropoxy]quinoline sesquifumarate, [U.S. Pat. No. 5,405,843 (continuation of 5,112,817)], [ACS # 158681-49-3], Sato et al., “Reversal of multidrug resistance by a novel quinoline derivative, MS-209, Cancer Chemother Pharmacol 35:271-277 (1995)), MS-073 (Fukazawa et al., European Patent Application 0363212 (1989)), FK-506 (Tanaka et al., M. Physicochemical properties of FK-506, a novel immunosuppressant isolated from Streptomyces tsukubaensis” Transplantation Proceedings. 19(5 Suppl 6): 11-6, (1987); Naito et al., “Reversal of multidrug resistance by an immunosuppressive agent FK-506,” Cancer Chemother & Pharmacol. 29:195-200 (1992); Pourtier-Manzanedo et al., “FK-506 (fujimycin) reverses the multidrug resistance of tumor cells in vitro,” Anti-Cancer Drugs 2:279-83 (1991); Epand & Epand, “The new potent immunosuppressant FK-506 reverses multidrug resistance in Chinese hamster ovary cells,” Anti-Cancer Drug Design 6:189-93 (1991)), VX-710 (2-peperidinecarboxylic acid, 1-[oxo(3,4,5-trimethoxyphenyl)acetyl]-3-(3-pyridinyl)-1-[3-(3-pyridinyl)propyl]butyl ester [ACS 159997-94-1] [U.S. Pat. No. 5,620,971] Germann et al., “Chemosensitization and drug accumulation effects of VX-710, verapamil, cyclosporin A, MS-209 and GF120918 in multidrug resistance-associated protein MRP” Anti-Cancer Drugs 8, 141-155 (1997), Germann et al, “Cellular and biochemical characterization of VX-710 as a chemosensitizer: reversal of β-glycoprotein-mediated multidrug resistance in vitro” Anti-Cancer Drugs 8, 125-140 (1997)), VX-853 ([U.S. Pat. No. 5,543,423] [ACS # 190454-58-1], AHC-52 (methyl 2-(N-benzyl-N-methylamino)ethyl-2, 6-dimethyl-4-(2-isopropylpyrazolo[1,5-a]pyridine-3-yl)-1,4-dihyropyridine-3,5-dicarboxylate; [Japanese Patent 63-135381; European Patent 0270926] [ACS 119666-09-0] Shinoda et al., “In vivo circumvention of vincristine resistance in mice with P388 leukemia using a novel compound, AHC-52,” Cancer Res 49:1722-6 (1989)), GF-120918 (9,10-dihydro-5-methoxy-9-oxo-N-[4-[2-(1,2,3,4-tetrahydro-6,7-dimethoxyisoquinol-2-yl) ethyl]phenyl]-4 acridinecarboxamide,[U.S. Pat. No. 5,604,237] [ACS # 143664-11-3] Hyafil et al., “In vitro and in vivo reversal of multidrug resistance by GF120918, an acridonecarboxamide derivative,” Cancer Res 53:4595-4602 (1993)), and XR-9051 ( 3-[(3Z, 6Z)-6-Benzylidene-1-methyl-2,5-dioxopiperazin-3-ylidenemethyl]-N-[4-[2-(6,7-dimethoxy-1,2,3,4-tetrahydroisoquinolin-2-yl)ethyl]phenyl]benzamide hydrochloride, [ACS#57-22-7]).

It is also within the scope of this invention to determine the ability of a compound (e.g., an ABCG4 transporter substrate) to interact with ABCG4 transporter without the labeling of any of the interactants. For example, a microphysiometer can be used to detect the interaction of a compound with ABCG4 transporter without the labeling of either the compound or the ABCG4. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and ABCG4.

In another embodiment, an assay is a cell-based assay comprising contacting a cell expressing an ABCG4 transporter target molecule (e.g., an ABCG4 transporter substrate) with a test compound and determining the ability of the test compound to modulate (e.g. stimulate or inhibit) the activity of the ABCG4 transporter target molecule. Determining the ability of the test compound to modulate the activity of an ABCG4 transporter target molecule can be accomplished, for example, by determining the ability of the ABCG4 transporter protein to bind to or interact with the ABCG4 transporter target molecule.

Determining the ability of the ABCG4 transporter protein or a biologically active fragment thereof, to bind to or interact with an ABCG4 transporter target molecule can be accomplished by one of the methods described above for determnining direct binding. In a preferred embodiment, determining the ability of the ABCG4 transporter protein to bind to or interact with an ABCG4 transporter target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target an appropriate substrate, detecting the induction of a reporter gene (comprising a target-responsive regulatory element operatively linked to a nucleic acid encoding a detectable marker, e.g., luciferase), cellular transport of, e.g., a reference compound or, e.g., a neurotoxic polypeptide (e.g., β-amyloid) or detecting a target-regulated cellular response.

In yet another embodiment, an assay of the present invention is a cell-free assay in which an ABCG4 transporter protein or biologically active portion thereof is contacted with a test compound and the ability of the test compound to bind to the ABCG4 transporter protein or biologically active portion thereof is determined. Preferred biologically active portions of the ABCG4 transporter proteins to be used in assays of the present invention include fragments which participate in interactions with non-ABCG4 transporter molecules, e.g., fragments with high surface probability scores. Binding of the test compound to the ABCG4 transporter protein can be determined either directly or indirectly as described above. In a preferred embodiment, the assay includes contacting the ABCG4 transporter protein or biologically active portion thereof with a known compound which binds ABCG4 transporter to form an assay mixture, contacting the assay mixture with a test compound, and determining the ability of the test compound to interact with an ABCG4 transporter protein, wherein determining the ability of the test compound to interact with an ABCG4 transporter protein comprises determining the ability of the test compound to preferentially bind to ABCG4 transporter or biologically active portion thereof as compared to the known compound.

In another embodiment, the assay is a cell-free assay in which an ABCG4 transporter protein or biologically active portion thereof is contacted with a test compound and the ability of the test compound to modulate (e.g., stimulate or inhibit) the activity of the ABCG4 transporter protein or biologically active portion thereof is determined. Determining the ability of the test compound to modulate the activity of an ABCG4 transporter protein can be accomplished, for example, by determining the ability of the ABCG4 transporter protein to bind to an ABCG4 transporter target molecule by one of the methods described above for determining direct binding. Alternatively, for example, ATP binding can be measured. Determining the ability of the ABCG4 transporter protein to bind to an ABCG4 transporter target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BlAcore). Changes in the optical phenomenon of surface plasmon resonance (SPR) can be used as an indication of real-time reactions between biological molecules.

In an alternative embodiment, determining the ability of the test compound to modulate the activity of an ABCG4 transporter protein can be accomplished by determining the ability of the ABCG4 transporter protein to further modulate the activity of a downstream effector of an ABCG4 transporter target molecule. For example, the activity of the effector molecule on an appropriate target can be determined or the binding of the effector to an appropriate target can be determined as previously described.

In yet another embodiment, the cell-free assay involves contacting an ABCG4 transporter protein or biologically active portion thereof with a known compound which binds the ABCG4 transporter protein to form an assay mixture, contacting the assay mixture with a test compound, and determining the ability of the test compound to interact with the ABCG4 transporter protein, wherein determining the ability of the test compound to interact with the ABCG4 transporter protein comprises determining the ability of the ABCG4 transporter protein to preferentially bind to or modulate the activity of an ABCG4 transporter target molecule.

The cell-free assays of the present invention are amenable to use of both soluble and/or membrane-bound forms of isolated proteins (e.g., ABCG4 transporter proteins or biologically active portions thereof ). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucainide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl═N,N-dimethyl-3-ammonio-l-propane sulfonate.

In more than one embodiment of the above assay methods of the present invention, it may be desirable to immobilize either ABCG4 transporter or its target molecule to facilitate separation of complexed from uncomplexed forms of one or both of the proteins, as well as to accommodate automation of the assay. Binding of a test compound to an ABCG4 transporter protein, or interaction of an ABCG4 transporter protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/ABCG4 transporter fusion proteins or glutathione-S-transferase/target fusion proteins can be adsorbed onto glutathione Sepharose beads (Sigma Chemical, St. Louis, Mo.) or glutathione derivatized microtitre plates, which are then combined with the test compound or the test compound and either the non-adsorbed target protein or ABCG4 transporter protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of ABCG4 transporter binding or activity determined using standard techniques.

Other techniques for immobilizing proteins on matrices can also be used in the screening assays of the invention. For example, either an ABCG4 transporter protein or an ABCG4 transporter target molecule can be immobilized utilizing conjugation of biotin and streptavidin. Biotinylated ABCG4 transporter protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with ABCG4 transporter protein or target molecules but which do not interfere with binding of the ABCG4 transporter protein to its target molecule can be derivatized to the wells of the plate, and unbound target or ABCG4 transporter protein trapped in the wells by antibody conjugation. Methods for detecting such complexes, in addition to those described above for the GST-immobilized complexes, include immunodetection of complexes using antibodies reactive with the ABCG4 transporter protein or target molecule, as well as enzyme-linked assays which rely on detecting an enzymatic activity associated with the ABCG4 transporter protein or target molecule.

In another embodiment, modulators of ABCG4 transporter expression are identified in a method wherein a cell is contacted with a candidate compound and the expression of ABCG4 transporter mRNA or protein in the cell is determined. The level of expression of ABCG4 transporter mRNA or protein in the presence of the candidate compound is compared to the level of expression of ABCG4 transporter mRNA or protein in the absence of the candidate compound. The candidate compound can then be identified as a modulator of ABCG4 transporter expression based on this comparison. For example, when expression of ABCG4 transporter mRNA or protein is greater (statistically significantly greater) in the presence of the candidate compound than in its absence, the candidate compound is identified as a stimulator of ABCG4 transporter mRNA or protein expression. Alternatively, when expression of ABCG4 transporter mRNA or protein is less (statistically significantly less) in the presence of the candidate compound than in its absence, the candidate compound is identified as an inhibitor of ABCG4 transporter mRNA or protein expression. The level of ABCG4 transporter mRNA or protein expression in the cells can be determined by methods described herein for detecting ABCG4 transporter mRNA or protein.

In yet another aspect of the invention, the ABCG4 transporter proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent W094/10300), to identify other proteins, which bind to or interact with ABCG4 transporter (“ABCG4-binding proteins” or “ABCG4-bp”) and are involved in ABCG4 transporter activity. Such ABCG4-binding proteins are also likely to be involved in the propagation of signals by the ABCG4 transporter proteins or ABCG4 transporter targets as, for example, downstream elements of an ABCG4-mediated signaling pathway. Alternatively, such ABCG4-binding proteins are likely to be ABCG4 transporter inhibitors.

The two-hybrid system is based on the modular nature of most transcription factors, which consist of separable DNA-binding and activation domains. Briefly, the assay utilizes two different DNA constructs. In one construct, the gene that codes for an ABCG4 transporter protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an ABCG4-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the ABCG4 transporter protein.

In another aspect, the invention pertains to a combination of two or more of the assays described herein. For example, a modulating agent can be identified using a cell-based or a cell free assay, and the ability of the agent to modulate the activity of an ABCG4 transporter protein can be confirmed in vivo, e.g., in an animal.

This invention further pertains to novel agents identified by the above-described screening assays. Accordingly, it is within the scope of this invention to further use an agent identified as described herein in an appropriate animal model. For example, an agent identified as described herein (e.g., an ABCG4 transporter modulating agent, an antisense ABCG4 transporter nucleic acid molecule, an ABCG4-specific antibody, or an ABCG4-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

2. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

a. Chromosome Mapping

Once the sequence (or a portion of the sequence) of a gene has been isolated, this sequence can be used to map the location of the gene on a chromosome. This process is called chromosome mapping. Accordingly, portions or fragments of the ABCG4 transporter nucleotide sequences, described herein, can be used to map the location of the ABCG4 transporter genes on a chromosome. The mapping of the ABCG4 transporter sequences to chromosomes is an important first step in correlating these sequences with genes associated with disease.

Briefly, ABCG4 transporter genes can be mapped to chromosomes by preparing PCR primers (preferably 15-25 bp in length) from the ABCG4 transporter nucleotide sequences. Computer analysis of the ABCG4 transporter sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the ABCG4 transporter sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a fuill set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the ABCG4 transporter nucleotide sequences to design oligonucleotide primers, sublocalization can be achieved with panels of fragments from specific chromosomes. Other mapping strategies which can similarly be used to map an ABCG4 transporter sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of BABC transporter Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data (such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the ABCG4 transporter gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

b. Tissue Typing

The ABCG4 transporter sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the ABCG4 transporter nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The ABCG4 transporter nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals.

If a panel of reagents from ABCG4 transporter nucleotide sequences described herein is used to generate a unique identification database for an individual, those same reagents can later be used to identify tissue from that individual. Using the unique identification database, positive identification of the individual, living or dead, can be made from extremely small tissue samples.

c. Use of ABCG4 Transporter Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NOs: 1 or 12 are particularly appropriate for this use as greater numbers of polymorphisms occur in the noncoding regions, making it easier to differentiate individuals using this technique. Examples of polynucleotide reagents include the ABCG4 transporter nucleotide sequences or portions thereof, e.g., fragments derived from the noncoding regions of SEQ ID NOs: 1 or 12 having a length of at least 20 bases, preferably at least 30 bases.

The ABCG4 transporter nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such ABCG4 transporter probes can be used to identify tissue by species and/or by organ type.

In a similar fashion, these reagents, e.g., ABCG4 transporter primers or probes can be used to screen tissue culture for contamination (i.e. screen for the presence of a mixture of different types of cells in a culture).

3. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining ABCG4 transporter protein and/or nucleic acid expression as well as ABCG4 transporter activity, in the context of a biological sample (e.g., blood, serum, cells, tissue) to thereby determine whether an individual is afflicted with a disease or disorder, or is at risk of developing a disorder, associated with aberrant or unwanted ABCG4 transporter expression or activity. The invention also provides for prognostic (or predictive) assays for determining whether an individual is at risk of developing a disorder associated with ABCG4 transporter protein, nucleic acid expression or activity. For example, mutations in an ABCG4 transporter gene can be assayed in a biological sample. Such assays can be used for prognostic or predictive purpose to thereby prophylactically treat an individual prior to the onset of a disorder characterized by or associated with ABCG4 transporter protein, nucleic acid expression or activity.

Another aspect of the invention pertains to monitoring the influence of agents (e.g., drugs, compounds) on the expression or activity of ABCG4 transporter in clinical trials.

These and other agents are described in further detail in the following sections.

a. Diagnostic Assays

An exemplary method for detecting the presence or absence of ABCG4 transporter protein or nucleic acid in a biological sample involves obtaining a biological sample from a test subject and contacting the biological sample with a compound or an agent capable of detecting ABCG4 transporter protein or nucleic acid (e.g., MRNA, genomic DNA) that encodes ABCG4 transporter protein such that the presence of ABCG4i transporter protein or nucleic acid is detected in the biological sample. The term “biological sample” is intended to include tissues, cells and biological fluids isolated from a subject, as well as tissues, cells and fluids present within a subject. A preferred agent for detecting ABCG4 transporter mRNA or genomic DNA is a labeled nucleic acid probe capable of hybridizing to ABCG4 transporter mRNA or genomic DNA. The nucleic acid probe can be, for example, a full-length ABCG4 transporter nucleic acid, such as the nucleic acid of SEQ ID NOs: 1 or 12, or a portion thereof, such as an oligonucleotide of at least 15, 30, 50, 100, 250 or 500 nucleotides in length and sufficient to specifically hybridize under stringent conditions to ABCG4 transporter mRNA or genomic DNA. Other suitable probes for use in the diagnostic assays of the invention are described herein.

A preferred agent for detecting ABCG4 transporter protein is an antibody capable of binding to ABCG4 transporter protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The detection method of the invention can be used to detect ABCG4 transporter mRNA, protein, or genomic DNA in a biological sample in vitro as well as in vivo. For example, in vitro techniques for detection of ABCG4 transporter mRNA include Northern hybridizations and in situ hybridizations. In vitro techniques for detection of ABCG4 transporter protein include enzyme linked immunosorbent assays (ELISAs), Western blots, immunoprecipitations and immunofluorescence. In vitro techniques for detection of ABCG4 transporter genomic DNA include Southern hybridizations. Furthermore, in vivo techniques for detection of ABCG4 transporter protein include introducing into a subject a labeled anti-ABCG4 transporter antibody. For example, the antibody can be labeled with a radioactive marker whose presence and location in a subject can be detected by standard imaging techniques.

In one embodiment, the methods further involve obtaining a control biological sample from a control subject, contacting the control sample with a compound or agent capable of detecting ABCG4 transporter protein, mRNA, or genomic DNA, such that the presence of ABCG4 transporter protein, mRNA or genomic DNA is detected in the biological sample, and comparing the presence of ABCG4 transporter protein, mRNA or genomic DNA in the control sample with the presence of ABCG4 transporter protein, mRNA or genomic DNA in the test sample.

The invention also encompasses kits for detecting the presence of ABCG4 transporter in a biological sample. For example, the kit can comprise a labeled compound or agent capable of detecting ABCG4 transporter protein or mRNA in a biological sample; means for determining the amount of ABCG4 transporter in the sample; and means for comparing the amount of ABCG4 transporter in the sample with a standard. The compound or agent can be packaged in a suitable container. The kit can further comprise instructions for using the kit to detect an ABCG4 transporter protein or nucleic acid.

b. Prognostic Assays

The diagnostic methods described herein can furthermore be utilized to identify subjects having or at risk of developing a disease or disorder associated with aberrant or unwanted ABCG4 transporter expression or activity. As used herein, the term “aberrant” includes an ABCG4 transporter expression or activity which deviates from the wild type ABCG4 transporter expression or activity. Aberrant expression or activity includes increased or decreased expression or activity, as well as expression or activity which does not follow the wild type developmental pattern of expression or the subcellular pattern of expression. For example, aberrant ABCG4 transporter expression or activity is intended to include the cases in which a mutation in the ABCG4 transporter gene causes the ABCG4 transporter gene to be under-expressed or over-expressed and situations in which such mutations result in a non-finctional ABCG4 transporter protein or a protein which does not function in a wild-type fashion, e.g., a protein which does not interact with an ABCG4 transporter ligand or one which interacts with a non-ABCG4 transporter ligand. As used herein, the term “unwanted” includes an unwanted phenomenon involved in a biological response. For example, the term “unwanted” includes an ABCG4 transporter expression or activity which is undesirable in a subject.

The assays described herein, such as the preceding diagnostic assays or the following assays, can be utilized to identify a subject having or at risk of developing a disorder associated with a misregulation in ABCG4 transporter protein activity or nucleic acid expression. Alternatively, the prognostic assays can be utilized to identify a subject having or at risk for developing a disorder associated with a misregulation in ABCG4 transporter protein activity or nucleic acid expression. Thus, the present invention provides a method for identifying a disease or disorder associated with aberrant or unwanted ABCG4 transporter expression or activity in which a test sample is obtained from a subject and ABCG4 transporter protein or nucleic acid (e.g., mRNA or genomic DNA) is detected, wherein the presence of ABCG4 transporter protein or nucleic acid is diagnostic for a subject having or at risk of developing a disease or disorder associated with aberrant or unwanted ABCG4 transporter expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted ABCG4 transporter expression or activity, e.g., a cancer where the cells of the cancer have developed multidrug resistance. Thus, the present invention provides methods for determining whether a subject can be effectively treated with an agent for a disorder associated with aberrant or unwanted ABCG4 transporter expression or activity in which a test sample is obtained and ABCG4 transporter protein or nucleic acid expression or activity is detected (e.g., wherein the abundance of ABCG4 transporter protein or nucleic acid expression or activity is diagnostic for a subject that can be administered the agent to treat a disorder associated with aberrant or unwanted ABCG4 transporter expression or activity).

The methods of the invention can also be used to detect genetic alterations (also referred to as “allelic variation”) in an ABCG4 transporter gene. In one embodiment, the method comprises the following two steps: (1) obtaining from a sample a polynucleotide that hybridizes to the human ABCG4 transporter gene (i.e., SEQ ID NO: 1), and (2) determining whether the polynucleotide is identical to a portion, or the full length sequence, of SEQ ID NO:I. In another embodiment, the method is used to determine if a subject with the altered gene is at risk for a disorder characterized by misregulation in ABCG4 transporter protein activity or nucleic acid expression. In preferred embodiments, the methods include detecting, in a sample of cells from the subject, the presence or absence of a genetic alteration characterized by at least one of an alteration affecting the integrity of a gene encoding an ABCG4 protein, or the mis-expression of the ABCG4 transporter gene. For example, such genetic alterations can be detected by ascertaining the existence of at least one of 1) a deletion of one or more nucleotides from an ABCG4 transporter gene; 2) an addition of one or more nucleotides to an ABCG4 transporter gene; 3) a substitution of one or more nucleotides of an ABCG4 transporter gene, 4) a chromosomal rearrangement of an ABCG4 transporter gene; 5) an alteration in the level of a messenger RNA transcript of an ABCG4 transporter gene, 6) aberrant modification of an ABCG4 transporter gene, such as of the methylation pattern of the genomic DNA, 7) the presence of a non-wild type splicing pattern of a messenger RNA transcript of an ABCG4 transporter gene, 8) a non-wild type level of an ABCG4-protein, 9) allelic loss of an ABCG4 transporter gene, and 10) inappropriate post-translational modification of an ABCG4-protein. As described herein, there are a large number of assays known in the art which can be used for detecting alterations in an ABCG4 transporter gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the ABCG4-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to an ABCG4 transporter gene under conditions such that hybridization and amplification of the ABCG4-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al, (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an ABCG4 transporter gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in ABCG4 transporter can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2:753-759). For example, genetic mutations in ABCG4 transporter can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the ABCG4 transporter gene and detect mutations by comparing the sequence of the sample ABCG4 transporter with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al (1996) Adv. Chromatogr. 36:127-162; and Griffin et al. (1993) Appl. Biochem. BiotechnoL 38:147-159).

Other methods for detecting mutations in the ABCG4 transporter gene include methods in which protection from cleavage agents is used to detect mismatched bases in RNA/RNA or RNA/DNA heteroduplexes (Myers et al (1985) Science 230:1242). In general, the art technique of “mismatch cleavage” starts by providing heteroduplexes formed by hybridizing (labeled) RNA or DNA containing the wild-type ABCG4 transporter sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defmed systems for detecting and mapping point mutations in ABCG4 transporter cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an ABCG4 transporter sequence, e.g., a wild-type ABCG4 transporter sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in ABCG4 transporter genes. For example, single strand conformation polymorphism (SSCP) may be used to detect differences in electrophoretic mobility between mutant and wild type nucleic acids (Orita et al (1989) Proc Natl. Acad. Sci USA: 86:2766, see also Cotton (1993) Mutat. Res. 285:125-144; and Hayashi (1992) Genet. Anal. Tech. Appi. 9:73-79). Single-stranded DNA fragments of sample and control ABCG4 transporter nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g. in clinical settings to diagnose patients exhibiting symptoms or family history of a disease or illness involving an ABCG4 transporter gene.

Furthermore, any cell type or tissue in which ABCG4 transporter is expressed may be utilized in the prognostic assays described herein.

c. Monitoring of Effects During Clinical Trials

Monitoring the influence of agents (e.g., drugs) on the expression or activity of an ABCG4 transporter protein can be applied not only in ABCG4 transporter drug screening, but also in clinical trials. For example, the effectiveness of an agent determined by a screening assay as described herein to increase ABCG4 transporter gene expression, protein levels, or upregulate ABCG4 transporter activity, can be monitored in clinical trials of subjects exhibiting decreased ABCG4 transporter gene expression, protein levels, or downregulated ABCG4 transporter activity. Alternatively, the effectiveness of an agent determined by a screening assay to decrease ABCG4 transporter gene expression, protein levels, or downregulate ABCG4 transporter activity, can be monitored in clinical trials of subjects exhibiting increased ABCG4 transporter gene expression, protein levels, or upregulated ABCG4 transporter activity. In such clinical trials, the expression or activity of an ABCG4 transporter gene, and preferably, other genes that have been implicated in, for example, an ABCG4-associated disorder can be used as a “read out” or markers of the phenotype of a particular cell.

For example, and not by way of limitation, genes, including ABCG4, that are modulated in cells by treatment with an agent (e.g., compound, drug or small molecule) which modulates ABCG4 transporter activity (e.g., identified in a screening assay as described herein) can be identified. Thus, to study the effect of agents on ABCG4-associated disorders, for example, in a clinical trial, cells can be isolated and RNA prepared and analyzed for the levels of expression of ABCG4 transporter and other genes implicated in the ABCG4-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of ABCG4 transporter or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an ABCG4 transporter protein, mRNA, or genomic DNA in the preadministration sample; (iii) obtaining one or more post-administration samples from the subject; (iv) detecting the level of expression or activity of the ABCG4 transporter protein, mRNA, or genomic DNA in the post-administration samples; (v) comparing the level of expression or activity of the ABCG4 transporter protein, mRNA, or genomic DNA in the pre-administration sample with the ABCG4 transporter protein, mRNA, or genomic DNA in the post administration sample or samples; and (vi) altering the administration of the agent to the subject accordingly. For example, increased administration of the agent may be desirable to increase the expression or activity of ABCG4 transporter to higher levels than detected, i.e., to increase the effectiveness of the agent. Alternatively, decreased administration of the agent may be desirable to decrease expression or activity of ABCG4 transporter to lower levels than detected, i.e. to decrease the effectiveness of the agent. According to such an embodiment, ABCG4 transporter expression or activity may be used as an indicator of the effectiveness of an agent, even in the absence of an observable phenotypic response.

4. Methods of Treatment:

The present invention provides for both prophylactic and therapeutic methods of treating a subject at risk of (or susceptible to) a disorder or having a disorder associated with aberrant or unwanted ABCG4 transporter expression or activity. With regards to both prophylactic and therapeutic methods of treatment, such treatments may be specifically tailored or modified, based on knowledge obtained from the field of phannacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the ABCG4 transporter molecules of the present invention or ABCG4 transporter modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

a. Prophylactic Methods

In one aspect, the invention provides a method for preventing in a subject, a disease or condition associated with an aberrant or unwanted ABCG4 transporter expression or activity, by administering to the subject an ABCG4 transporter or an agent which modulates ABCG4 transporter expression or at least one ABCG4 transporter activity. Subjects at risk for a disease which is caused or contributed to by aberrant or unwanted ABCG4 transporter expression or activity can be identified by, for example, any or a combination of diagnostic or prognostic assays as described herein. Administration of a prophylactic agent can occur prior to the manifestation of symptoms characteristic of the ABCG4 transporter aberrancy, such that a disease or disorder is prevented or, alternatively, delayed in its progression. Depending on the type of ABCG4 transporter aberrancy, for example, an ABCG4, ABCG4 transporter agonist or A3CG4 transporter antagonist agent can be used for treating the subject. The appropriate agent can be determined based on screening assays described herein.

b. Therapeutic Methods

Another aspect of the invention pertains to methods of modulating ABCG4 transporter expression or activity for therapeutic purposes. Accordingly, in an exemplary embodiment, the modulatory method of the invention involves contacting a cell with an ABCG4 transporter or agent that modulates one or more of the activities of ABCG4 transporter protein activity associated with the cell. An agent that modulates ABCG4 transporter protein activity can be an agent as described herein, such as a nucleic acid or a protein, a naturally-occurring target molecule of an ABCG4 transporter protein (e.g., an ABCG4 transporter substrate), an ABCG4 transporter antibody, an ABCG4 transporter agonist or antagonist, a peptidomimetic of an ABCG4 transporter agonist or antagonist, or other small molecule. In one embodiment, the agent stimulates one or more ABCG4 transporter activities. Examples of such stimulatory agents include active ABCG4 transporter protein and a nucleic acid molecule encoding an ABCG4 transporter that has been introduced into the cell. In another embodiment, the agent inhibits one or more ABCG4 transporter activities. Examples of such inhibitory agents include antisense ABCG4 transporter nucleic acid molecules, anti-ABCG4 transporter antibodies, and ABCG4 transporter inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an ABCG4 transporter protein or nucleic acid molecule. In one embodiment, the method involves administering an agent (e.g., an agent identified by a screening assay described herein), or combination of agents that modulates (e.g., upregulates or downregulates) ABCG4 transporter expression or activity. In another embodiment, the method involves administering an ABCG4 transporter protein or nucleic acid molecule as therapy to compensate for reduced, aberrant, or unwanted ABCG4 transporter expression or activity.

Stimulation of ABCG4 transporter activity is desirable in situations in which ABCG4 transporter is abnormally downregulated and/or in which increased ABCG4 transporter activity is likely to have a beneficial effect. For example, stimulation of ABCG4 transporter activity is desirable in situations in which an ABCG4 transporter is downregulated and/or in which increased ABCG4 transporter activity is likely to have a beneficial effect. Likewise, inhibition of ABCG4 transporter activity is desirable in situations in which ABCG4 transporter is abnormally upregulated and/or in which decreased ABCG4 transporter activity is likely to have a beneficial effect.

In one embodiment, an agent found to inhibit ABCG4 transporter activity is used in combination with another therapy such that the targeting of that therapy across the blood-brain-barrier is achieved.

c. Pharmacogenomics

The ABCG4 transporter molecules of the present invention, as well as agents, or modulators which have a stimulatory or inhibitory effect on ABCG4 transporter activity (e.g., ABCG4 transporter gene expression) as identified by a screening assay described herein can be administered to individuals to treat (prophylactically or therapeutically) ABCG4-associated disorders associated with aberrant or unwanted ABCG4 transporter activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an ABCG4 transporter molecule or ABCG4 transporter modulator as well as tailoring the dosage and/or therapeutic regimen of treatment with an ABCG4 transporter molecule or ABCG4 transporter modulator.

Pharmacogenomics deals with clinically significant hereditary variations in the response to drugs due to altered drug disposition and abnormal action in affected persons. See, for example, Eichelbaum, M. et al (1996) Clin. Exp. Pharmacol. Physiol. 23(10-11) :983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase 11/111 drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an ABCG4 transporter protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-forned metabolite morphine. The other extreme are the so called ultrα-rapid metabolizers who do not respond to standard doses. Recently, the molecular basis of ultrα-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an ABCG4 transporter molecule or ABCG4 transporter modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an ABCG4 transporter molecule or ABCG4 transporter modulator, such as a modulator identified by one of the exemplary screening assays described herein.

This invention is further illustrated by the following examples which should not be construed as limiting.

### EXAMPLES

### Example 1

Isolation and Cloning of the Human ABCG4 Transpoter cDNA

In this example, the isolation and cloning of the gene encoding a novel human ABCG4 transporter is described.

Bioinformatics analysis of the genomic sequence AC000384 reveals a putative open reading frame (ORF) in which the 3′ end matched the EST AL137563. Oligonucleotides were generated using the above-mentioned nucleotide sequences and used to produce a complementary DNA (cDNA) fragment. It is a 3455 base pair (bp) fragment obtained by reverse transcriptase polymerase chain reaction (RTPCR) from human brain total RNA using oligonucleotides PI and P2 (defined below). The cDNA sequence was obtained using a LICOR automated DNA sequencing engine. The sequencing data identified a 1941 nucleotide fragment that contains the complete open reading frame of the invention. By comparison to the nucleotide and amino acid sequence data banks, the deduced amino acid sequence of the invention reveals a unique human protein. The closest protein homologs are ABCGl, ABCG2, ABCG5, ABCG8 (80%, 47%, 44% and 43% similarity, respectively), members of the ATβ-binding cassette (ABC) transporter superfamily (gene nomenclature approved by the Human Genome Organization (http://www.gene.ucl.ac.uk/nomenclature/)) (Table 1). The percentage of nucleotide identity between the isolated ABCG4 and its closest homologs according to Pairwise Global Aligment (MacVector 7.0) is shown in Table 2. The ABC transporter superfamily is divided into several groups in which proteins share common structural features. Therefore human ABCG4 transporter is a new ABC transporter that belongs to the G group.

The following provides a detailed description for the generation of the PCR fragments:

RT-PCR

1 μg of normal human Brain mRNA (Invitrogen) was subjected to first strand synthesis according to Life Tech's cDNA ThermoScript RT-PCR System (Cat. No. 11146-024). The DNA was then diluted 1 to 10 with Tricine-EDTA buffer and an aliquot was submitted to PCR using the following primers:

Oligonucleotides

Sequences of the oligonucleotides used in the RT-PCR reaction:

| | | |
|---|---|---|
| 5′ - GCCACCATGGCGGAGAAGGCGCTGGAG - 3′ | P1 (1-27) | (SEQ ID NO:14) |
| 5′ - CCCACAAATACAACTGGCATCCCTG - 3′ | P2 (3431-3455) | (SEQ ID NO:15) |

PCR Conditions

The PCR reaction was carried out using Clontech Advantage cDNA PCR kit (#K1905-1) in a 50 μl final volume using 2.5 μl of the diluted first strand cDNA and primer P1 and P2 according to the manufacturer's instruction. The cycling parameters were: denaturing at 94° C. for 1 min followed by 30 cycles of denaturing at 94° C. for 10 sec, annealing at 60° C. for 30 sec, elongating at 72° C. for 5 min.

TABLE 1
Percentage of amino acid identity and similarity
between ABCG4 and its closest homologs
(identity/similarity) according to Pairwise
Global Alignment (MacVector 7.0)

| | | | | CAC- | | |
|---|---|---|---|---|---|---|
| | ABCG1 | ABCG2 | ABCG4 | 17140 | ABCG5 | ABCG8 |
| ABCG1 | 100/100 | 27/20 | 67/13 | 67/12 | 21/23 | 21/22 |
| (SEQ ID | | | | | | |
| NO: 4; Fig. | | | | | | |
| 4A-B) | | | | | | |
| ABCG2 | 27/20 | 100/100 | 26/21 | 26/21 | 24/23 | 23/19 |
| (SEQ ID | | | | | | |
| NO: 5; Fig. | | | | | | |
| 4A-B) | | | | | | |
| ABCG4 | 67/13 | 26/21 | 100/100 | 95/0 | 23/22 | 21/20 |
| CAC17140 | 67/12 | 26/21 | 95/0 | 100/100 | 23/22 | 21/21 |
| (SEQ ID | | | | | | |
| NO: 8; Fig. | | | | | | |
| 5) | | | | | | |
| ABCG5 | 21/23 | 24/23 | 23/22 | 23/22 | 100/100 | 26/19 |
| (SEQ ID | | | | | | |
| NO: 6; Fig. | | | | | | |
| 4A-B) | | | | | | |
| ABCG8 | 21/22 | 23/19 | 21/20 | 21/21 | 26/19 | 100/100 |
| (SEQ ID | | | | | | |
| NO: 7; Fig. | | | | | | |
| 4A-B) | | | | | | |

TABLE 2
Percentage of nucleotide identity between
ABCG4 and its closest homologs at the
According to Pairwise Global Alignment (MacVector 7.0)

| | ABCG1 | ABCG2 | ABCG4 | AJ300465 | ABCG5 | ABCG8 |
|---|---|---|---|---|---|---|
| ABCG1 | 100 | 33 | 50 | 49 | 30 | 27 |
| ABCG2 | 33 | 100 | 27 | 26 | 28 | 30 |
| ABCG4 | 50 | 27 | 100 | 98 | 25 | 23 |
| AJ300465 | 49 | 26 | 98 | 100 | 25 | 23 |
| ABCG5 | 30 | 28 | 25 | 25 | 100 | 36 |
| ABCG8 | 27 | 30 | 23 | 23 | 36 | 100 |

### Example 2

Characterization of the Novel Human ABCG4 Transporter Molecule

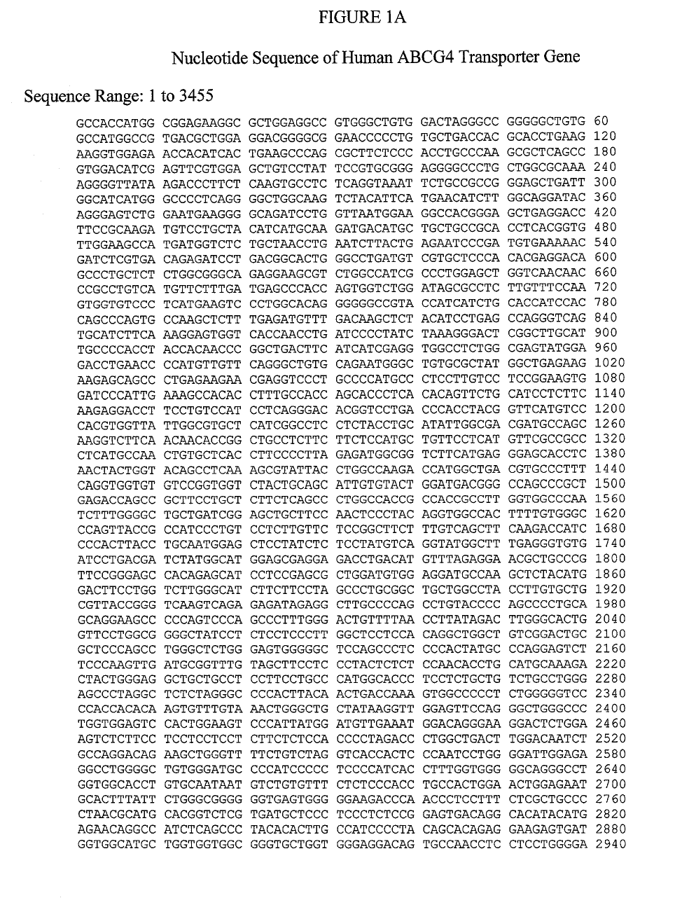

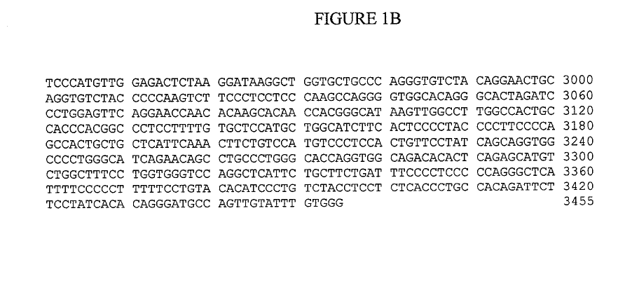

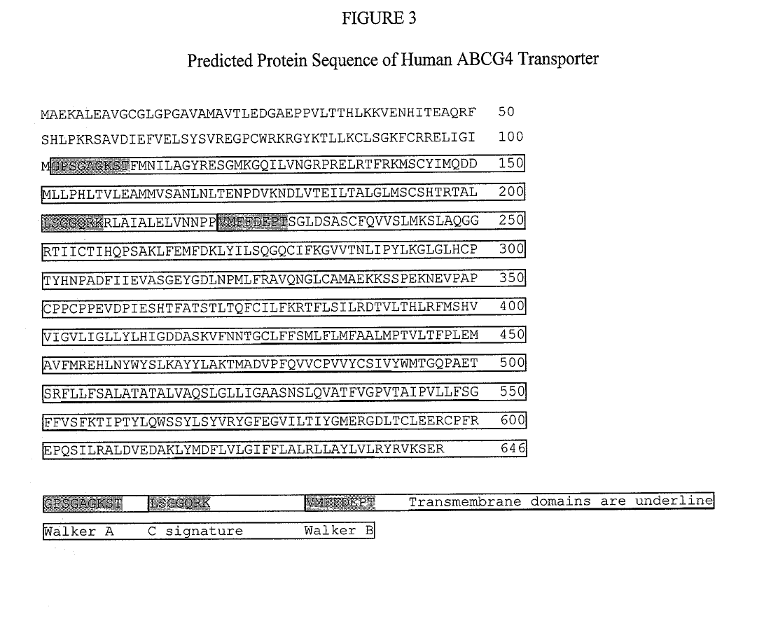

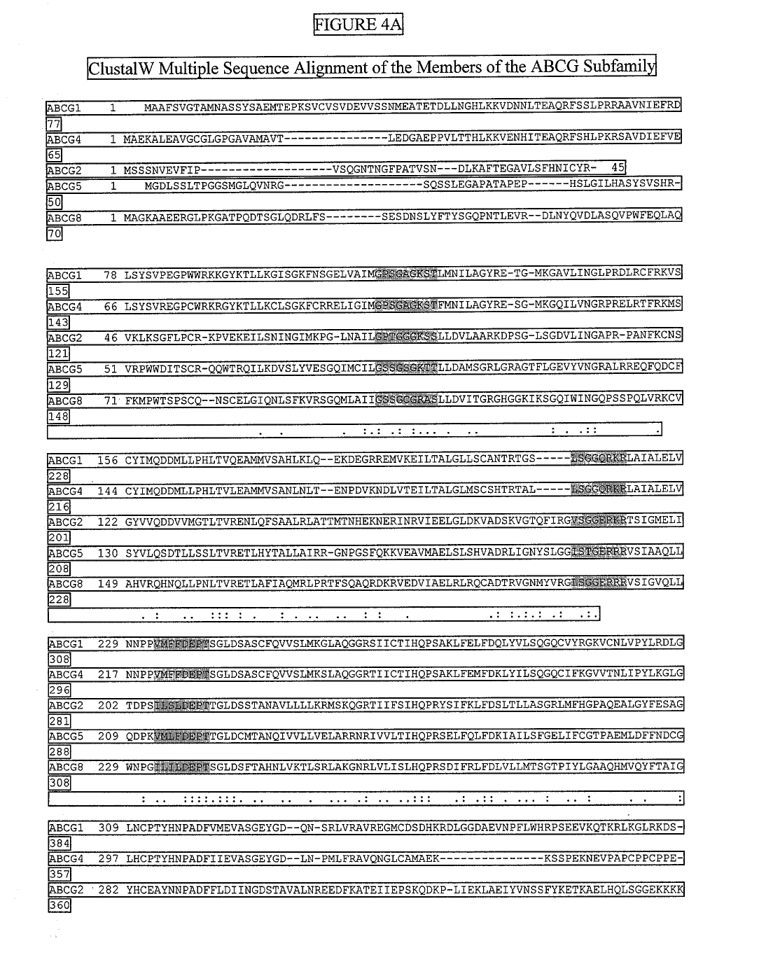

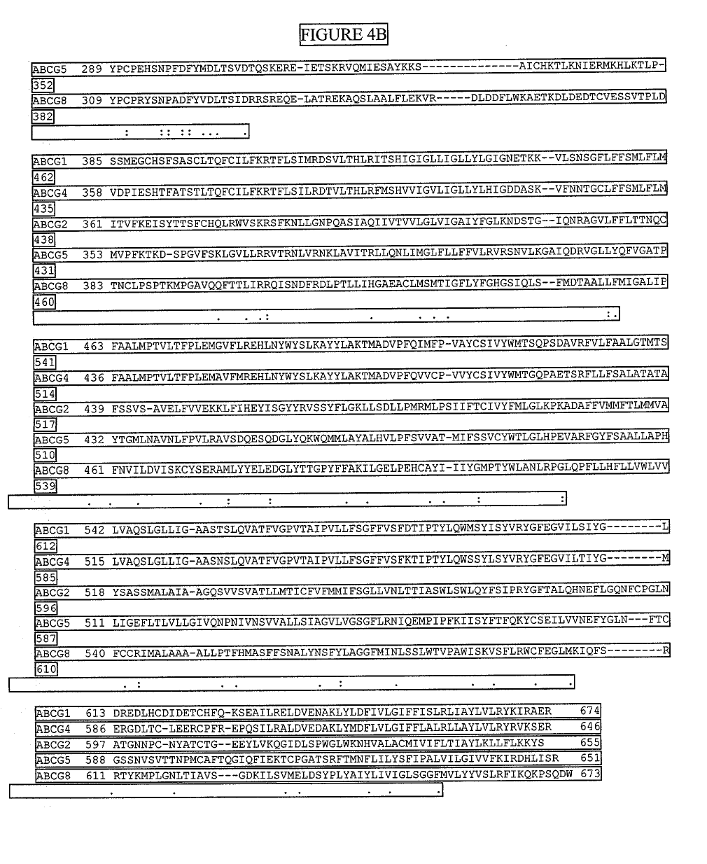

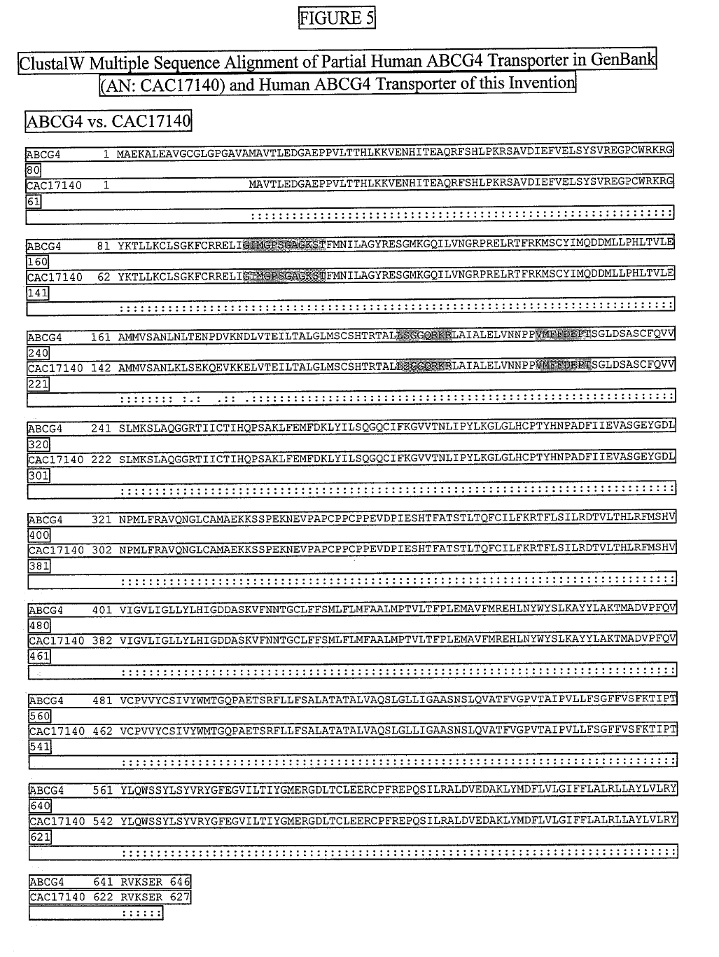

The longest open reading frame of the ABCG4 nucleic acid is a 1941 nucleotide sequence that begins with the sequence ACCATGG that matches the consensus eukaryotic translation initiation motif at nucleotide position 7 (ATG) and ends with a TAG termination signal at position 1945, the cDNA sequence of which is disclosed in SEQ ID 10 NO:1, with the corresponding protein sequence disclosed in SEQ ID NO:2 (FIGS. 1A-B and FIG. 3, respectively). If the first in-frame ATG encodes the amino terminal methionine, a deduced polypeptide of 645 amino acids with a predicted molecular weight of 71.8 kDa would result. The predicted peptide was analyzed in respect to potential membrane spanning segments. Hydropathy plots and 2D fold models obtained using 15 bioinformatics tools (PredictProtein server, Heidelberg, Germany) defined seven putative membrane spanning regions in the C-terminal sequence of the protein, all of them having a helical 2D fold structure, which suggest that human ABCG4 is a membrane anchored protein. The N-terminal sequence contains multiple clusters of conserved residues that define an ATP binding domain and ABC transporter signature motif (FIG. 3). Therefore, the ABCG4 protein exhibits the typical features conserved among ABC transporters. ABC transporter proteins structural organization represents variations on common themes. One common theme is the basic structure of those transporters with twelve hydrophobic transmembrane segments and two hydrophilic ATP binding sites, either present in a single polypeptide chain or assembled from half or quarter molecules. Therefore the putative amino acid sequence of ABCG4 defines it as a novel hemitransporter. It is likely but not necessary that the ABCG4 protein would function as part of a dimeric transporter structure. To date the few hemitransporters characterized in mammalian cells have been found to be localized to the membrane of intracellular compartments. Bioinformatics modeling tools propose that the N-terminal part as well as the ATP binding cassette of the predicted protein could be in an “outside” conformation. It then would appear likely but not necessary that human ABCG4 could be associated with an intracellular membrane structure rather than with the cytoplasmic membrane.

### Example 3

Expression of a Recombinant Human ABCG4 Transporter Polypedptide in Mammalian Cells

A recombinant clone was obtained where the ABCG4 ORF was subcloned in frame with an artificial nucleotide sequence into a mammalian expression vector (PCEP4, Invitrogen Corp, CA, USA, #V380-20). The construct allowed the expression of a recombinant protein where the ABCG4 peptidique sequence was fused with a fourteen amino acid peptide (epitope tag, Invitrogen, #K48000-01) to facilitate detection of the expressed chimera in vitro. Transient transfection of HEK293 cells with the clone pCEPG4CV5 led to transient expression of a 70 kDa protein.

### Example 4

Expression of Wild Type, but not Mutant ABCG4 Gene Increasses Both Intracellular APP and Extracellular Aβ Levels

This example shows that the expression of the wild type ABCG4 gene, but not genes encoding mutant ABCG4 proteins, increases both cellular APP and secreted Aβ levels in cells transfected with either a wild type APP gene or a Swedish mutant APβ-695 gene.

Methods

Cell Lines and Transient Transfections

293 EBNA cells (InVitrogen, Carlsbad, CA) stably transfected with a gene encoding a wild-type or a Swedish mutant Amyloid Precursor Protein-695 (WT-6 and SM4 cells, respectively) were routinely maintained in DMEM supplemented with sodium pyruvate (1 mM) and 10% fetal bovine serum. Cells were seeded on Poly-D-Lysine (SIGMA) coated 35 mm² plates at a density of 1.25×10⁵ cells/plate 18 hours prior to transient transfection experiments. Cultures were then transfected for 48 hrs using a Fugene-6 transfection procedure (Boehringer Mannheim, Laval, QC) that entailed the use of 2 μg DNA at a DNA:Fugene-6 ratio of 1:3. Control cultures were transfected with β-galactosidase (in the pCEP4 vector) while treated cultures were transfected with ABCG4 or one of three ABCG4 mutants altered at the Walker A and/or Walker B motifs (G4mA: K→R, G4mB: D→N, G4mAB: K→R and D→N) subeloned into pCEP4. After the 48 hour transfection period, the cells were rinsed with 1 ml of warm PBS (37° C.), and the WT-6 cells were exposed in 1 ml serum free DMEM supplemented with sodium pyruvate (1 mM) for 4 hours, while the SM-4 cells were exposed in serum free DMEM supplemented with sodium pyruvate (1 mM) for 16 hrs. Transfection efficiency was monitored in each experiment using β-galactosidase (β-gal) staining kit (Invitrogen, Carlsbad, Calif.).

APP Detection

To measure intracellular Amyloid Precursor Protein (APP) levels, cells were harvested with ice-cold lysis buffer and sonicated on ice for 8 seconds using a probe sonicator. From each sample, total protein concentration was determined using the bicinchonic acid assay (Pierce, Rockford, Ill., USA). Cellular APP levels were quantitated by 10% Tris-Glyine SDS-PAGE Western blot analysis using an anti-APP N-terminal antibody (22C11, Boehringer Mannhein, Laval, QC) (Mills et al., 1997; Connop et al., 1999). Immunoreactive bands were visualized using ECL detection (Amersham, Oakville, ON) and analyzed by standard densitometric techniques.

Aβ Detection

For the WT-6 cells, culture media was harvested after 4 hr exposure and subjected to trichloroacetic acid precipitation. The pellet was resuspended in Tris-Tricine SDS sample buffer (6% SDS, 5% β-mercaptoethanol, 10% glycerol, 0.006% G-250, 188 mM Tris-HCl, pH 6.8) and normalized levels of protein were subjected to electrophoresis on a 16.5% Tris-Tricine sodium dodecyl sulfate gel as previously described (Mills et al., 1997). Total Aβ was measured using monoclonal antibody 6E10 (Senetek Research, Maryland Heights, Mo., USA). Bands were visualized using ECL detection (Amersham, Oakville, ON) and analyzed by standard densitometric techniques.

For the SM-4 cells, culture media was harvested after 16 hr exposure and supplemented with 10% sample treatment buffer (40 mM sodium phosphate (pH 7.4), 40 mM triethanolamine, 0.1% Triton X-100, 200 mM NaCl, 2mM EGTA, 0.1% Sodium azide), and assayed for either Aβ-40 or Aβ-42 by a colorimetric ELISA as per the manufacturer's protocol (Biosource International Inc, California). Amyloid levels were normalized to total cellular protein.

Statistical Analysis

Statistical significance was determined using an ANOVA with Tukey's post hoc analysis. Data are expressed as mean±SD with * p<0.05, **p<0.01 and ***p<0.001 and n=5.

Results

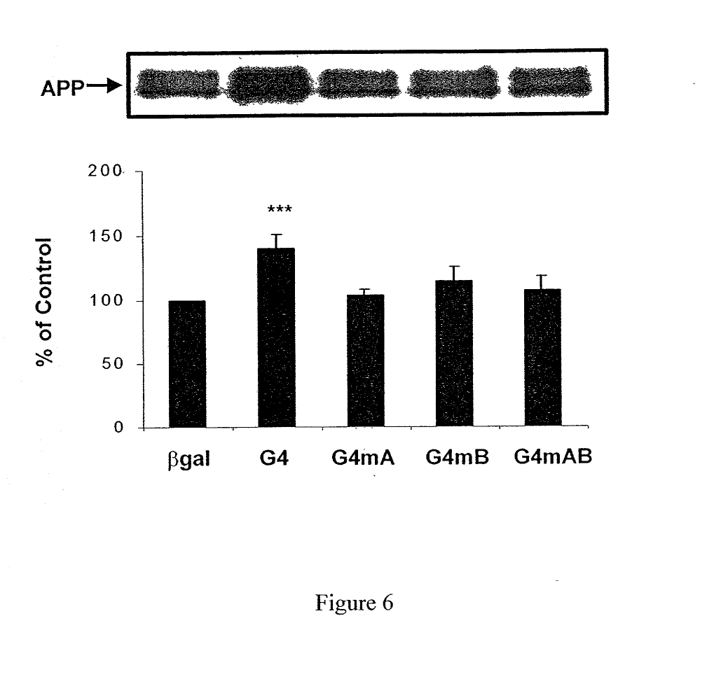

FIG. 6 shows cellular APP levels from WT6 transiently transfected with a gene encoding β-galactosidase, ABCG4 or one of three ABCG4 mutant proteins. After a 48 hr transfection interval, cells were incubated for 4 hrs and cellular APP was quantitated by Western blot analysis. A representative micrograph of the APP Western blot data is depicted above the corresponding densitometric values. Data are expressed as mean±SD with n=5 and statistical significance determined by ANOVA with Tukey's post hoc test at ***p<0.001.

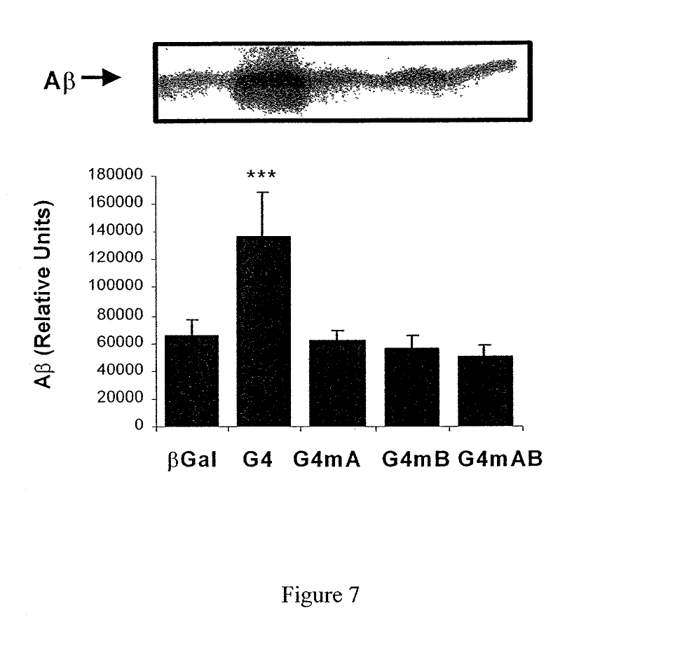

FIG. 7 shows Aβ release from WT6 cells transiently transfected with a gene encoding β-galactosidase, ABCG4 or one of three ABCG4 mutant proteins. After a 48 hr transfection interval, cells were incubated for 4 hrs and Aβ release was quantitated by Western blot analysis. A representative micrograph of the Aβ Western blot data is depicted above the corresponding densitometric values. Data are expressed as mean±SD with n=5 and statistical significance determined by ANOVA with Tukey's post hoc test at V ***p<0.001.

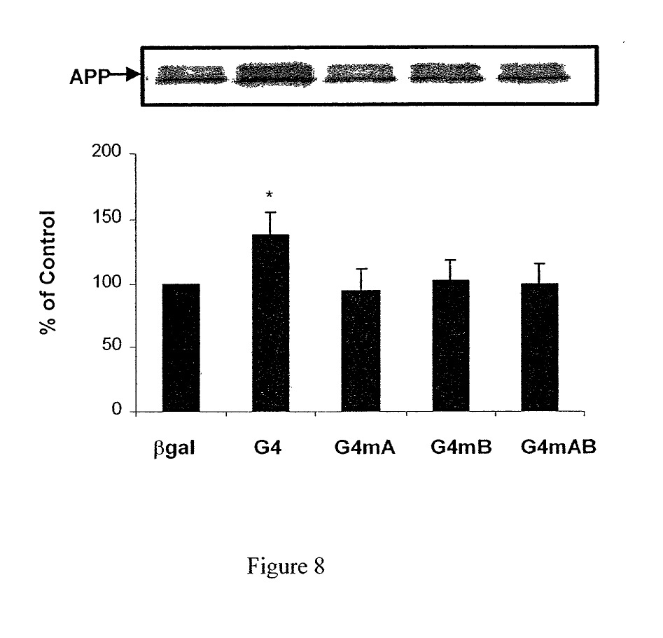

FIG. 8 shows cellular APP levels from SM4 cells transiently transfected with a gene encoding β-galactosidase, ABCG4 or one of three ABCG4 mutant proteins. After a 48 hr transfection interval, cells were incubated for 16 hrs and cellular APP was quantitated by Western blot analysis. Data are expressed as mean±SD with n=5 and statistical significance determined by ANOVA with Tukey's post hoc test at *p<0.05.

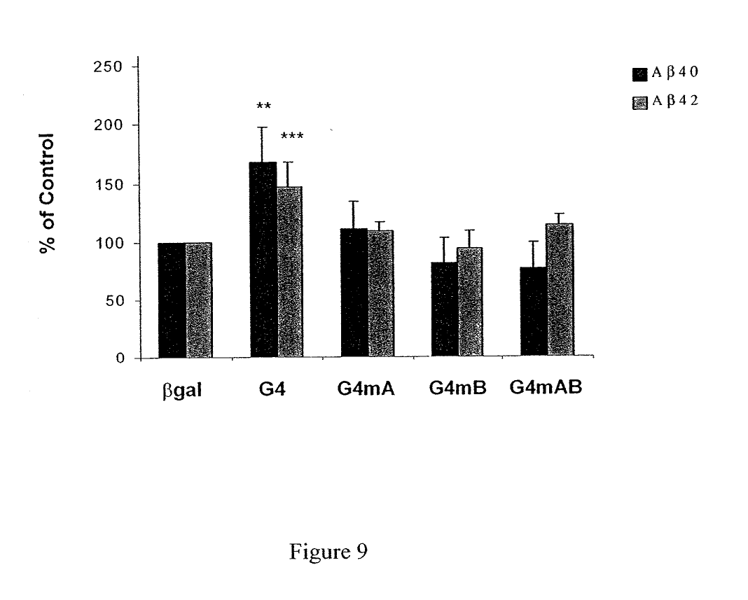

FIG. 9 shows Aβ release from SM4 cells transiently transfected with a gene encoding β-Galactosidase, ABCG4 or one of three ABCG4 mutant proteins. After a 48 hr transfection interval, cells were incubated for 16 hrs and Aβ-40 and Aβ-42 release were quantitated by ELISA. Data are expressed as mean±SD with n=5 and statistical significance determined by ANOVA with Tukey's post hoc test at **p<0.01 and ***p<0.001.

### Example 5

Isolation and Cloning of the Human ABCG4.2 Transporter cDNA

In this example, the isolation and cloning of a second cDNA sequence encoding a novel human ABCG4 transporter is described.

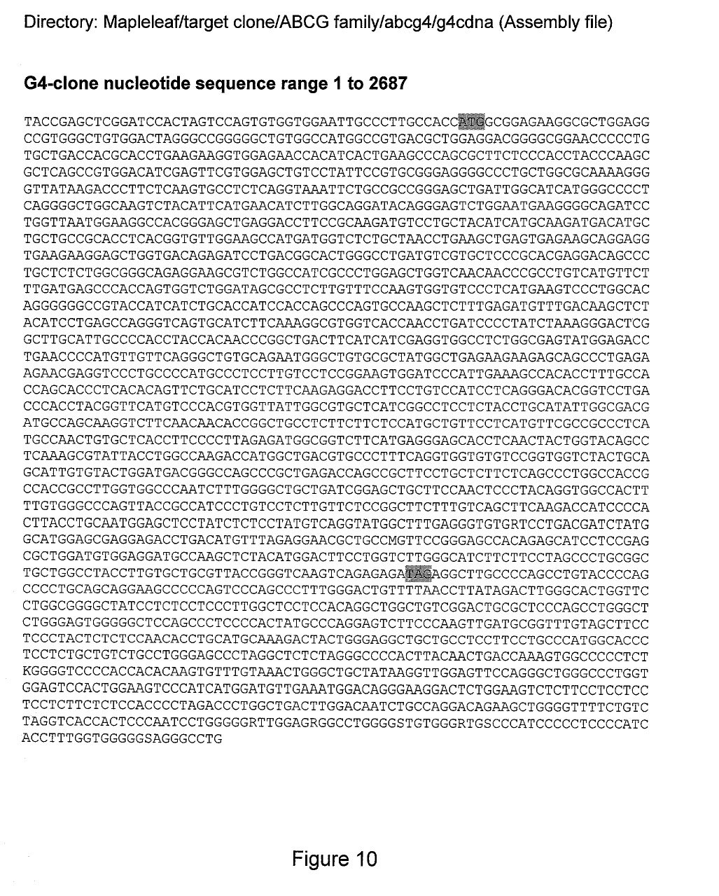

Bioinformatics analysis of the genomic sequence AC000384 reveals a putative open reading frame (ORF) in which the 3′ end matched the EST AL137563. Oligonucleotides were generated using the above-mentioned nucleotide sequences and used to produce a complementary DNA (cDNA) fragment. It is a 2687 base pair (bp) fragment (SEQ ID NO:12: FIG. 10) obtained by reverse transcriptase polymerase chain reaction (RTPCR) from human brain total RNA using oligonucleotides P3 and P4 (SEQ ID NOs:16 and 17, respectively, defined below). The cDNA sequence was obtained using a LICOR automated DNA sequencing engine. The sequencing data identified a 1941 nucleotide fragment that contains the complete open reading frame of the invention. By comparison to the nucleotide and amino acid sequence data banks, the deduced amino acid sequence of the invention reveals a unique human protein. The closest protein homologs are ABCG1, ABCG2, ABCG5, ABCG8 (80%, 47%, 44% and 43% similarity, respectively), members of the ATβ-binding cassette (ABC) transporter superfamily (gene nomenclature approved by the Human Genome Organization (http://www.gene.ucl.ac.uk/nomenclature/)) (Table 3). The percentage of nucleotide identity between the isolated ABCG4 and its closest homologs according to Pairwise Global Aligment (MacVector 7.0) is shown in Table 4. The ABC transporter superfamily is divided into several groups in which proteins share common structural features. Therefore human ABCG4 transporter is a new ABC transporter that belongs to the G group.

The following provides a detailed description for the generation of the PCR fragments:

RT-PCR

1 μg of normal human Brain mRNA (Invitrogen) was subjected to first strand synthesis according to Life Tech's cDNA ThernoScript RT-PCR System (Cat. No. 11146-024). The DNA was then diluted 1 to 10 with Tricine-EDTA buffer and an aliquot was submitted to PCR using the following primers:

Oligonucleotides

Sequences of the oligonucleotides used in the RT-PCR reaction:

| | | |
|---|---|---|
| 5′ - TACCGAGCTCGGATCCACTAGTCC - 3′ | P3 (1-24) | (SEQ ID NO:16) |
| 5′ - CAGGCCCTGCCCCCACCAAAGGT - 3′ | P4 (2665-2687) | (SEQ ID NO:17) |

PCR Conditions

The PCR reaction was carried out using Clontech Advantage cDNA PCR kit (#K1905-1) in a 50 μl final volume using 2.5 μl of the diluted first strand cDNA and primer P3 and P4 according to the manufacturer's instruction. The cycling parameters were: denaturing at 94° C. for 1 min followed by 30 cycles of denaturing at 94° C. for 10 sec, annealing at 60° C. for 30 sec, elongating at 72° C. for 5 min.

TABLE 3
Percentage of amino acid identity and
similarity between ABCG4.2 and its closest
homologs (identity/similarity) according to
Pairwise Global Alignment (MacVector 7.0)

| | ABCG1 | | | CAC- | | |
|---|---|---|---|---|---|---|
| | variant I | ABCG2 | ABCG4.2 | 17140 | ABCG5 | ABCG8 |
| ABCG1 | 100/100 | 27/20 | 67/13 | 67/12 | 21/23 | 21/22 |
| variant I | | | | | | |
| ABCG2 | 27/20 | 100/100 | 26/21 | 26/21 | 24/23 | 23/19 |
| ABCG4.2 | 67/13 | 26/21 | 100/100 | 95/0 | 23/22 | 21/20 |
| CAC17140 | 67/12 | 26/21 | 95/0 | 100/100 | 23/22 | 21/21 |
| ABCG5 | 21/23 | 24/23 | 23/22 | 23/22 | 100/100 | 26/19 |
| ABCG8 | 21/22 | 23/19 | 21/20 | 21/21 | 26/19 | 100/100 |

TABLE 4
Percentage of nucleotide identity between
ABCG4.2 and its closest homologs at the
According to Pairwise Global Alignment (MacVector 7.0)

| | ABCG1 | ABCG2 | ABCG4.2 | AJ300465 | ABCG5 | ABCG8 |
|---|---|---|---|---|---|---|
| ABCG1 | 100 | 33 | 50 | 49 | 30 | 27 |
| ABCG2 | 33 | 100 | 27 | 26 | 28 | 30 |
| ABCG4.2 | 50 | 27 | 100 | 98 | 25 | 23 |
| AJ300465 | 49 | 26 | 98 | 100 | 25 | 23 |
| ABCG5 | 30 | 28 | 25 | 25 | 100 | 36 |
| ABCG8 | 27 | 30 | 23 | 23 | 36 | 100 |

### Example 6

Characterization of the Novel Human ABCG4 Transporter Molecule

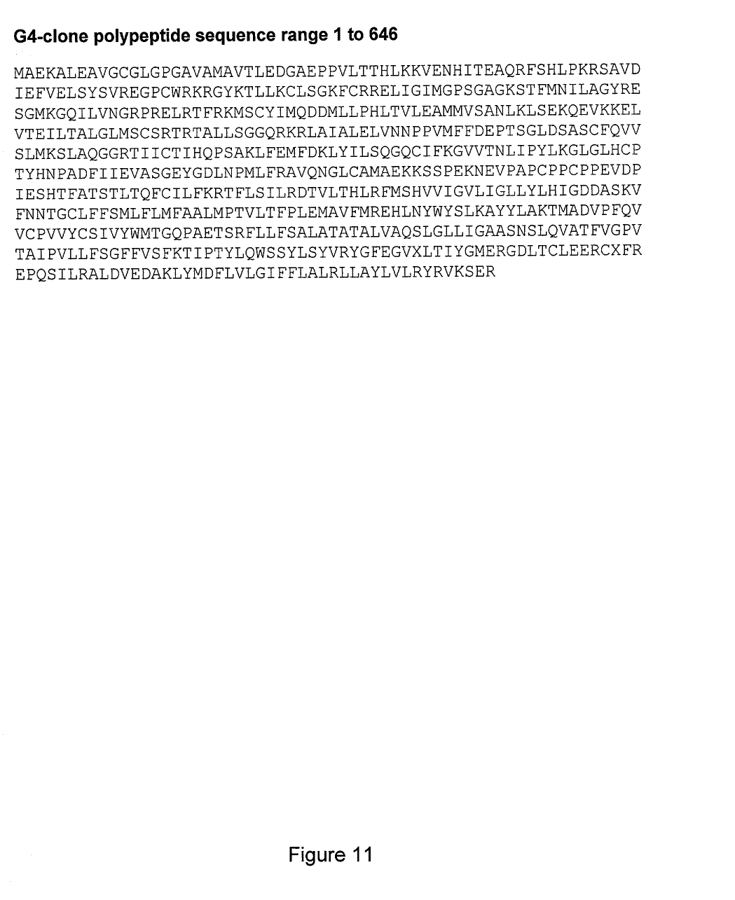

The longest open reading frame of the ABCG4.2 nucleic acid is a 1941 nucleotide sequence that begins with the sequence ACCATGG that matches the consensus eukaryotic translation initiation motif at nucleotide position 51 (ATG) and ends with a TAG termination signal at position 1989. If the first in-frame ATG encodes the amino terminal methionine, a deduced polypeptide of 646 amino acids (FIG. 11; SEQ ID NO:13) with a predicted molecular weight of 71.8 kDa would result. The predicted peptide was analyzed in respect to potential membrane spanning segments. Hydropathy plots and 2D fold models obtained using bioinformatics tools (PredictProtein server, Heidelberg, Germany) defined seven putative membrane spanning regions in the C-terminal sequence of the protein, all of them having a helical 2D fold structure, which suggest that human ABCG4.2 is a membrane anchored protein. The N-terminal sequence contains multiple clusters of conserved residues that define an ATP binding domain and ABC transporter signature motif (FIG. 3). Therefore, the ABCG4.2 protein exhibits the typical features conserved among ABC transporters. ABC transporter proteins structural organization represents variations on common themes. One common theme is the basic structure of those transporters with twelve hydrophobic transmembrane segments and two hydrophilic ATP binding sites, either present in a single polypeptide chain or assembled from half or quarter molecules. Therefore the putative amino acid sequence of ABCG4.2 defines it as a novel hemitransporter. It is likely but not necessary that the ABCG4.2 protein would function as part of a dimeric transporter structure. To date the few hemitransporters characterized in mammalian cells have been found to be localized to the membrane of intracellular compartments. Bioinformatics modeling tools propose that the N-terminal part as well as the ATP binding cassette of the predicted protein could be in an “outside” conformation. It then would appear likely but not necessary that human ABCG4.2 could be associated with an intracellular membrane structure rather than with the cytoplasmic membrane.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated nucleic acid molecule selected from the group consisting of:
(a) a nucleic acid molecule comprising a nucleotide sequence which encodes a polypeptide comprising the amino acid sequence set forth in SEQ ID NOs: 2 or 13; and
(b) a nucleic acid molecule comprising a nucleotide sequence which encodes a naturally occurring allelic variant of a polypeptide comprising the amino acid sequence set forth in SEQ ID NOs: 2 or 13, wherein said allelic variant binds to an antibody that selectively binds to the polypeptide of SEQ ID NOs: 2 or 13, and is not the polypeptide within the amino acid sequence of SEQ ID NO: 4.

2. The isolated nucleic acid molecule of claim 1 comprising the nucleotide sequence set forth in SEQ ID NOs: 1, 3 or 12.

3. An isolated nucleic acid molecule comprising a nucleotide sequence which is complementary to the nucleotide sequence of the nucleic acid molecule of claim 1.

4. An isolated nucleic acid molecule comprising the nucleic acid molecule of claim 1 and a nucleotide sequence encoding a heterologous polypeptide.

5. A vector comprising the nucleic acid molecule of claim l.

6. The vector of claim 6, which is an expression vector.

7. A host cell transfected with the expression vector of claim 6.

8. A method of producing a polypeptide comprising culturing the host cell of claim 7 in an appropriate culture medium to thereby produce the polypeptide.

9. An isolated polypeptide selected from the group consisting of:
(a) a polypeptide comprising the amino acid sequence of SEQ ID NOs: 2 or 13; and
(b) a polypeptide comprising a naturally occurring allelic variant of a polypeptide comprising the amino acid sequence set forth in SEQ ID NOs: 2 or 13, wherein said allelic variant binds to an antibody that selectively binds to the polypeptide of SEQ ID NOs: 2 or 13, and is not the polypeptide with the amino acid sequence of SEQ ID NO: 4.

10. The polypeptide of claim 9, further comprising at least one heterologous amino acid sequence at the amino- and/or the carboxyl-terminus of said polypeptide.

11. An antibody which selectively binds to a polypeptide of claim 9.

12. A method for detecting the presence of a polypeptide of claim 9 in a biological sample comprising:
(a) contacting said sample with a compound which selectively binds to the polypeptide; and
(b) detecting the presence of a complex between said compound and said polypeptide.

13. The method of claim 12, wherein the compound which binds to the polypeptide is an antibody.

14. A kit comprising a compound which selectively binds to a polypeptide of claim 9 and instructions for use.

15. A method for detecting the presence of a nucleic acid molecule of claim 1 in a biological sample comprising:
(a) contacting said sample with a nucleic acid probe or primer which selectively hybridizes to the nucleic acid molecule; and
(b) detecting the presence of a complex of said nucleic acid molecule and said probe or primer.

16. The method of claim 15, wherein the sample comprises mRNA molecules and is contacted with a nucleic acid probe.

17. A kit comprising a compound which selectively hybridizes to a nucleic acid molecule of claim 1 and instructions for use.

18. A method for identifying a compound which binds to a polypeptide of claim 9 comprising:
(a) contacting said polypeptide, or a cell expressing said polypeptide with a test compound; and
(b) determining whether the polypeptide binds to the test compound.

19. The method of claim 18, wherein the binding of the test compound to the polypeptide is detected by a method selected from the group consisting of:
(a) direct detection of binding;
(b) detection of binding using a competition binding assay; and
(c) detection of binding using an assay for ABCG4 transporter activity.

20. A method for modulating the activity of a polypeptide of claim 9 comprising contacting said polypeptide or a cell expressing said polypeptide with a compound which binds to the polypeptide in a sufficient concentration to modulate the activity of the polypeptide.

21. A method for identifying a compound which modulates the activity of a polypeptide of claim 9 comprising:
(a) contacting a polypeptide of claim 9 with a test compound; and
(b) determining the effect of the test compound on the activity of the polypeptide to thereby identify a compound which modulates the activity of the polypeptide.

22. A method for detecting an allelic variant of the nucleic acid of SEQ ID NOs: 1 or 12 or an orthologue thereof in a biological sample, comprising:
(a) obtaining from the sample a polynucleotide that hybridizes to the nucleic acid of SEQ ID NO:lI or the orthologue thereof; and
(b) determining whether said polynulclotide is identical to a portion, or the fuill length sequence, of SEQ ID NOs: 1 or 12, or the orthologue thereof.

23. A composition comprising a pharmaceutically effective amount of the nucleic acid molecule of SEQ ID NOs: 1 or 12 and a pharmaceutically acceptable carrier.

24. A composition comprising a pharmaceutically effective amount of an antisense oligonucleotide capable of specifically hybridizing to the nucleic acid sequence of SEQ ID NOs: 1 or 12 and a pharmaceutically acceptable carrier.

25. A transgenic knockout mouse whose genome comprises a homozygous disruption in its endogenous ABCO4 gene, wherein said homozygous disruption prevents the expression of a fuinctional ABCG4 protein, and wherein said homozygous disruption results in said transgenic knockout mouse being sterile.


SEQUENCE LISTING (17 sequences)

SEQ ID NO: 1 - DNA, 3455 bp, Homo sapiens
gccaccatggcggagaaggcgctggaggccgtgggctgtggactagggccgggggctgtggccatggccgtgacgctggaggacggggcggaaccccctgtgctgaccacgcacctgaagaaggtggagaaccacatcactgaagcccagcgcttctcccacctgcccaagcgctcagccgtggacatcgagttcgtggagctgtcctattccgtgcgggaggggccctgctggcgcaaaaggggttataagacccttctcaagtgcctctcaggtaaattctgccgccgggagctgattggcatcatgggcccctcaggggctggcaagtctacattcatgaacatcttggcaggatacagggagtctggaatgaaggggcagatcctggttaatggaaggccacgggagctgaggaccttccgcaagatgtcctgctacatcatgcaagatgacatgctgctgccgcacctcacggtgttggaagccatgatggtctctgctaacctgaatcttactgagaatcccgatgtgaaaaacgatctcgtgacagagatcctgacggcactgggcctgatgtcgtgctcccacacgaggacagccctgctctctggcgggcagaggaagcgtctggccatcgccctggagctggtcaacaacccgcctgtcatgttctttgatgagcccaccagtggtctggatagcgcctcttgtttccaagtggtgtccctcatgaagtccctggcacaggggggccgtaccatcatctgcaccatccaccagcccagtgccaagctctttgagatgtttgacaagctctacatcctgagccagggtcagtgcatcttcaaaggagtggtcaccaacctgatcccctatctaaagggactcggcttgcattgccccacctaccacaacccggctgacttcatcatcgaggtggcctctggcgagtatggagacctgaaccccatgttgttcagggctgtgcagaatgggctgtgcgctatggctgagaagaagagcagccctgagaagaacgaggtccctgccccatgccctccttgtcctccggaagtggatcccattgaaagccacacctttgccaccagcaccctcacacagttctgcatcctcttcaagaggaccttcctgtccatcctcagggacacggtcctgacccacctacggttcatgtcccacgtggttattggcgtgctcatcggcctcctctacctgcatattggcgacgatgccagcaaggtcttcaacaacaccggctgcctcttcttctccatgctgttcctcatgttcgccgccctcatgccaactgtgctcaccttccccttagagatggcggtcttcatgagggagcacctcaactactggtacagcctcaaagcgtattacctggccaagaccatggctgacgtgccctttcaggtggtgtgtccggtggtctactgcagcattgtgtactggatgacgggccagcccgctgagaccagccgcttcctgctcttctcagccctggccaccgccaccgccttggtggcccaatctttggggctgctgatcggagctgcttccaactccctacaggtggccacttttgtgggcccagttaccgccatccctgtcctcttgttctccggcttctttgtcagcttcaagaccatccccacttacctgcaatggagctcctatctctcctatgtcaggtatggctttgagggtgtgatcctgacgatctatggcatggagcgaggagacctgacatgtttagaggaacgctgcccgttccgggagccacagagcatcctccgagcgctggatgtggaggatgccaagctctacatggacttcctggtcttgggcatcttcttcctagccctgcggctgctggcctaccttgtgctgcgttaccgggtcaagtcagagagatagaggcttgccccagcctgtaccccagcccctgcagcaggaagcccccagtcccagccctttgggactgttttaaccttatagacttgggcactggttcctggcggggctatcctctcctcccttggctcctccacaggctggctgtcggactgcgctcccagcctgggctctgggagtgggggctccagccctccccactatgcccaggagtcttcccaagttgatgcggtttgtagcttcctccctactctctccaacacctgcatgcaaagactactgggaggctgctgcctccttcctgcccatggcaccctcctctgctgtctgcctgggagccctaggctctctagggccccacttacaactgaccaaagtggccccctctgggggtccccaccacacaagtgtttgtaaactgggctgctataaggttggagttccagggctgggccctggtggagtccactggaagtcccattatggatgttgaaatggacagggaaggactctggaagtctcttcctcctcctcctcttctctccacccctagaccctggctgacttggacaatctgccaggacagaagctgggttttctgtctaggtcaccactcccaatcctggggattggagaggcctggggctgtgggatgccccatccccctccccatcacctttggtgggggcagggcctggtggcacctgtgcaataatgtctgtgtttctctcccacctgccactggaactggagaatgcactttattctgggcggggggtgagtgggggaagacccaaccctcctttctcgctgcccctaacgcatgcacggtctcgtgatgctccctccctctccggagtgacaggcacatacatgagaacaggccatctcagccctacacacttgccatcccctacagcacagaggaagagtgatggtggcatgctggtggtggcgggtgctggtgggaggacagtgccaacctcctcctggggatcccatgttggagactctaaggataaggctggtgctgcccagggtgtctacaggaactgcaggtgtctacccccaagtcttccctcctcccaagccaggggtggcacagggcactagatccctggagttcaggaaccaacacaagcacaaccacgggcataagttggccttggccactgccacccacggccctccttttgtgctccatgctggcatcttcactcccctaccccttccccagccactgctgctcattcaaacttctgtccatgtccctccactgttcctatcagcaggtggcccctgggcatcagaacagcctgccctgggcaccaggtggcagacacactcagagcatgtctggctttcctggtgggtccaggctcattctgcttctgatttcccctcccccagggctcattttccccctttttcctgtacacatccctgtctacctcctctcaccctgccacagattcttcctatcacacagggatgccagttgtatttgtggg

SEQ ID NO: 2 - protein, 646 aa, Homo sapiens
MAEKALEAVGCGLGPGAVAMAVTLEDGAEPPVLTTHLKKVENHITEAQRFSHLPKRSAVDIEFVELSYSVREGPCWRKRGYKTLLKCLSGKFCRRELIGIMGPSGAGKSTFMNILAGYRESGMKGQILVNGRPRELRTFRKMSCYIMQDDMLLPHLTVLEAMMVSANLNLTENPDVKNDLVTEILTALGLMSCSHTRTALLSGGQRKRLAIALELVNNPPVMFFDEPTSGLDSASCFQVVSLMKSLAQGGRTIICTIHQPSAKLFEMFDKLYILSQGQCIFKGVVTNLIPYLKGLGLHCPTYHNPADFIIEVASGEYGDLNPMLFRAVQNGLCAMAEKKSSPEKNEVPAPCPPCPPEVDPIESHTFATSTLTQFCILFKRTFLSILRDTVLTHLRFMSHVVIGVLIGLLYLHIGDDASKVFNNTGCLFFSMLFLMFAALMPTVLTFPLEMAVFMREHLNYWYSLKAYYLAKTMADVPFQVVCPVVYCSIVYWMTGQPAETSRFLLFSALATATALVAQSLGLLIGAASNSLQVATFVGPVTAIPVLLFSGFFVSFKTIPTYLQWSSYLSYVRYGFEGVILTIYGMERGDLTCLEERCPFREPQSILRALDVEDAKLYMDFLVLGIFFLALRLLAYLVLRYRVKSER

SEQ ID NO: 3 - DNA, 1941 bp, Homo sapiens
atggcggagaaggcgctggaggccgtgggctgtggactagggccgggggctgtggccatggccgtgacgctggaggacggggcggaaccccctgtgctgaccacgcacctgaagaaggtggagaaccacatcactgaagcccagcgcttctcccacctgcccaagcgctcagccgtggacatcgagttcgtggagctgtcctattccgtgcgggaggggccctgctggcgcaaaaggggttataagacccttctcaagtgcctctcaggtaaattctgccgccgggagctgattggcatcatgggcccctcaggggctggcaagtctacattcatgaacatcttggcaggatacagggagtctggaatgaaggggcagatcctggttaatggaaggccacgggagctgaggaccttccgcaagatgtcctgctacatcatgcaagatgacatgctgctgccgcacctcacggtgttggaagccatgatggtctctgctaacctgaatcttactgagaatcccgatgtgaaaaacgatctcgtgacagagatcctgacggcactgggcctgatgtcgtgctcccacacgaggacagccctgctctctggcgggcagaggaagcgtctggccatcgccctggagctggtcaacaacccgcctgtcatgttctttgatgagcccaccagtggtctggatagcgcctcttgtttccaagtggtgtccctcatgaagtccctggcacaggggggccgtaccatcatctgcaccatccaccagcccagtgccaagctctttgagatgtttgacaagctctacatcctgagccagggtcagtgcatcttcaaaggagtggtcaccaacctgatcccctatctaaagggactcggcttgcattgccccacctaccacaacccggctgacttcatcatcgaggtggcctctggcgagtatggagacctgaaccccatgttgttcagggctgtgcagaatgggctgtgcgctatggctgagaagaagagcagccctgagaagaacgaggtccctgccccatgccctccttgtcctccggaagtggatcccattgaaagccacacctttgccaccagcaccctcacacagttctgcatcctcttcaagaggaccttcctgtccatcctcagggacacggtcctgacccacctacggttcatgtcccacgtggttattggcgtgctcatcggcctcctctacctgcatattggcgacgatgccagcaaggtcttcaacaacaccggctgcctcttcttctccatgctgttcctcatgttcgccgccctcatgccaactgtgctcaccttccccttagagatggcggtcttcatgagggagcacctcaactactggtacagcctcaaagcgtattacctggccaagaccatggctgacgtgccctttcaggtggtgtgtccggtggtctactgcagcattgtgtactggatgacgggccagcccgctgagaccagccgcttcctgctcttctcagccctggccaccgccaccgccttggtggcccaatctttggggctgctgatcggagctgcttccaactccctacaggtggccacttttgtgggcccagttaccgccatccctgtcctcttgttctccggcttctttgtcagcttcaagaccatccccacttacctgcaatggagctcctatctctcctatgtcaggtatggctttgagggtgtgatcctgacgatctatggcatggagcgaggagacctgacatgtttagaggaacgctgcccgttccgggagccacagagcatcctccgagcgctggatgtggaggatgccaagctctacatggacttcctggtcttgggcatcttcttcctagccctgcggctgctggcctaccttgtgctgcgttaccgggtcaagtcagagagatag

SEQ ID NO: 4 - protein, 674 aa, Homo sapiens
MAAFSVGTAMNASSYSAEMTEPKSVCVSVDEVVSSNMEATETDLLNGHLKKVDNNLTEAQRFSSLPRRAAVNIEFRDLSYSVPEGPWWRKKGYKTLLKGISGKFNSGELVAIMGPSGAGKSTLMNILAGYRETGMKGAVLINGLPRDLRCFRKVSCYIMQDDMLLPHLTVQEAMMVSAHLKLQEKDEGRREMVKEILTALGLLSCANTRTGSLSGGQRKRLAIALELVNNPPVMFFDEPTSGLDSASCFQVVSLMKGLAQGGRSIICTIHQPSAKLFELFDQLYVLSQGQCVYRGKVCNLVPYLRDLGLNCPTYHNPADFVMEVASGEYGDQNSRLVRAVREGMCDSDHKRDLGGDAEVNPFLWHRPSEEVKQTKRLKGLRKDSSSMEGCHSFSASCLTQFCILFKRTFLSIMRDSVLTHLRITSHIGIGLLIGLLYLGIGNETKKVLSNSGFLFFSMLFLMFAALMPTVLTFPLEMGVFLREHLNYWYSLKAYYLAKTMADVPFQIMFPVAYCSIVYWMTSQPSDAVRFVLFAALGTMTSLVAQSLGLLIGAASTSLQVATFVGPVTAIPVLLFSGFFVSFDTIPTYLQWMSYISYVRYGFEGVILSIYGLDREDLHCDIDETCHFQKSEAILRELDVENAKLYLDFIVLGIFFISLRLIAYLVLRYKIRAER

SEQ ID NO: 5 - protein, 655 aa, Homo sapiens
MSSSNVEVFIPVSQGNTNGFPATVSNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIEELGLDKVADSKVGTQFIRGVSGGERKRTSIGMELITDPSILSLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 6 - protein, 651 aa, Homo sapiens
MGDLSSLTPGGSMGLQVNRGSQSSLEGAPATAPEPHSLGILHASYSVSHRVRPWWDITSCRQQWTRQILKDVSLYVESGQIMCILGSSGSGKTTLLDAMSGRLGRAGTFLGEVYVNGRALRREQFQDCFSYVLQSDTLLSSLTVRETLHYTALLAIRRGNPGSFQKKVEAVMAELSLSHVADRLIGNYSLGGISTGERRRVSIAAQLLQDPKVMLFDEPTTGLDCMTANQIVVLLVELARRNRIVVLTIHQPRSELFQLFDKIAILSFGELIFCGTPAEMLDFFNDCGYPCPEHSNPFDFYMDLTSVDTQSKEREIETSKRVQMIESAYKKSAICHKTLKNIERMKHLKTLPMVPFKTKDSPGVFSKLGVLLRRVTRNLVRNKLAVITRLLQNLIMGLFLLFFVLRVRSNVLKGAIQDRVGLLYQFVGATPYTGMLNAVNLFPVLRAVSDQESQDGLYQKWQMMLAYALHVLPFSVVATMIFSSVCYWTLGLHPEVARFGYFSAALLAPHLIGEFLTLVLLGIVQNPNIVNSVVALLSIAGVLVGSGFLRNIQEMPIPFKIISYFTFQKYCSEILVVNEFYGLNFTCGSSNVSVTTNPMCAFTQGIQFIEKTCPGATSRFTMNFLILYSFIPALVILGIVVFKIRDHLISR

SEQ ID NO: 7 - protein, 673 aa, Homo sapiens
MAGKAAEERGLPKGATPQDTSGLQDRLFSSESDNSLYFTYSGQPNTLEVRDLNYQVDLASQVPWFEQLAQFKMPWTSPSCQNSCELGIQNLSFKVRSGQMLAIIGSSGCGRASLLDVITGRGHGGKIKSGQIWINGQPSSPQLVRKCVAHVRQHNQLLPNLTVRETLAFIAQMRLPRTFSQAQRDKRVEDVIAELRLRQCADTRVGNMYVRGLSGGERRRVSIGVQLLWNPGILILDEPTSGLDSFTAHNLVKTLSRLAKGNRLVLISLHQPRSDIFRLFDLVLLMTSGTPIYLGAAQHMVQYFTAIGYPCPRYSNPADFYVDLTSIDRRSREQELATREKAQSLAALFLEKVRDLDDFLWKAETKDLDEDTCVESSVTPLDTNCLPSPTKMPGAVQQFTTLIRRQISNDFRDLPTLLIHGAEACLMSMTIGFLYFGHGSIQLSFMDTAALLFMIGALIPFNVILDVISKCYSERAMLYYELEDGLYTTGPYFFAKILGELPEHCAYIIIYGMPTYWLANLRPGLQPFLLHFLLVWLVVFCCRIMALAAAALLPTFHMASFFSNALYNSFYLAGGFMINLSSLWTVPAWISKVSFLRWCFEGLMKIQFSRRTYKMPLGNLTIAVSGDKILSVMELDSYPLYAIYLIVIGLSGGFMVLYYVSLRFIKQKPSQDW

SEQ ID NO: 8 - protein, 627 aa, Homo sapiens
MAVTLEDGAEPPVLTTHLKKVENHITEAQRFSHLPKRSAVDIEFVELSYSVREGPCWRKRGYKTLLKCLSGKFCRRELIGIMGPSGAGKSTFMNILAGYRESGMKGQILVNGRPRELRTFRKMSCYIMQDDMLLPHLTVLEAMMVSANLKLSEKQEVKKELVTEILTALGLMSCSHTRTALLSGGQRKRLAIALELVNNPPVMFFDEPTSGLDSASCFQVVSLMKSLAQGGRTIICTIHQPSAKLFEMFDKLYILSQGQCIFKGVVTNLIPYLKGLGLHCPTYHNPADFIIEVASGEYGDLNPMLFRAVQNGLCAMAEKKSSPEKNEVPAPCPPCPPEVDPIESHTFATSTLTQFCILFKRTFLSILRDTVLTHLRFMSHVVIGVLIGLLYLHIGDDASKVFNNTGCLFFSMLFLMFAALMPTVLTFPLEMAVFMREHLNYWYSLKAYYLAKTMADVPFQVVCPVVYCSIVYWMTGQPAETSRFLLFSALATATALVAQSLGLLIGAASNSLQVATFVGPVTAIPVLLFSGFFVSFKTIPTYLQWSSYLSYVRYGFEGVILTIYGMERGDLTCLEERCPFREPQSILRALDVEDAKLYMDFLVLGIFFLALRLLAYLVLRYRVKSER

SEQ ID NO: 9 - protein, 9 aa, Homo sapiens
GPSGAGKST

SEQ ID NO: 10 - protein, 7 aa, Homo sapiens
LSGGQRK

SEQ ID NO: 11 - protein, 8 aa, Homo sapiens
VMFFDEPT

SEQ ID NO: 12 - DNA, 2687 bp, Homo sapiens
taccgagctcggatccactagtccagtgtggtggaattgcccttgccaccatggcggagaaggcgctggaggccgtgggctgtggactagggccgggggctgtggccatggccgtgacgctggaggacggggcggaaccccctgtgctgaccacgcacctgaagaaggtggagaaccacatcactgaagcccagcgcttctcccacctacccaagcgctcagccgtggacatcgagttcgtggagctgtcctattccgtgcgggaggggccctgctggcgcaaaaggggttataagacccttctcaagtgcctctcaggtaaattctgccgccgggagctgattggcatcatgggcccctcaggggctggcaagtctacattcatgaacatcttggcaggatacagggagtctggaatgaaggggcagatcctggttaatggaaggccacgggagctgaggaccttccgcaagatgtcctgctacatcatgcaagatgacatgctgctgccgcacctcacggtgttggaagccatgatggtctctgctaacctgaagctgagtgagaagcaggaggtgaagaaggagctggtgacagagatcctgacggcactgggcctgatgtcgtgctcccgcacgaggacagccctgctctctggcgggcagaggaagcgtctggccatcgccctggagctggtcaacaacccgcctgtcatgttctttgatgagcccaccagtggtctggatagcgcctcttgtttccaagtggtgtccctcatgaagtccctggcacaggggggccgtaccatcatctgcaccatccaccagcccagtgccaagctctttgagatgtttgacaagctctacatcctgagccagggtcagtgcatcttcaaaggcgtggtcaccaacctgatcccctatctaaagggactcggcttgcattgccccacctaccacaacccggctgacttcatcatcgaggtggcctctggcgagtatggagacctgaaccccatgttgttcagggctgtgcagaatgggctgtgcgctatggctgagaagaagagcagccctgagaagaacgaggtccctgccccatgccctccttgtcctccggaagtggatcccattgaaagccacacctttgccaccagcaccctcacacagttctgcatcctcttcaagaggaccttcctgtccatcctcagggacacggtcctgacccacctacggttcatgtcccacgtggttattggcgtgctcatcggcctcctctacctgcatattggcgacgatgccagcaaggtcttcaacaacaccggctgcctcttcttctccatgctgttcctcatgttcgccgccctcatgccaactgtgctcaccttccccttagagatggcggtcttcatgagggagcacctcaactactggtacagcctcaaagcgtattacctggccaagaccatggctgacgtgccctttcaggtggtgtgtccggtggtctactgcagcattgtgtactggatgacgggccagcccgctgagaccagccgcttcctgctcttctcagccctggccaccgccaccgccttggtggcccaatctttggggctgctgatcggagctgcttccaactccctacaggtggccacttttgtgggcccagttaccgccatccctgtcctcttgttctccggcttctttgtcagcttcaagaccatccccacttacctgcaatggagctcctatctctcctatgtcaggtatggctttgagggtgtgrtcctgacgatctatggcatggagcgaggagacctgacatgtttagaggaacgctgccmgttccgggagccacagagcatcctccgagcgctggatgtggaggatgccaagctctacatggacttcctggtcttgggcatcttcttcctagccctgcggctgctggcctaccttgtgctgcgttaccgggtcaagtcagagagatagaggcttgccccagcctgtaccccagcccctgcagcaggaagcccccagtcccagccctttgggactgttttaaccttatagacttgggcactggttcctggcggggctatcctctcctcccttggctcctccacaggctggctgtcggactgcgctcccagcctgggctctgggagtgggggctccagccctccccactatgcccaggagtcttcccaagttgatgcggtttgtagcttcctccctactctctccaacacctgcatgcaaagactactgggaggctgctgcctccttcctgcccatggcaccctcctctgctgtctgcctgggagccctaggctctctagggccccacttacaactgaccaaagtggccccctctkggggtccccaccacacaagtgtttgtaaactgggctgctataaggttggagttccagggctgggccctggtggagtccactggaagtcccatcatggatgttgaaatggacagggaaggactctggaagtctcttcctcctcctcctcttctctccacccctagaccctggctgacttggacaatctgccaggacagaagctggggttttctgtctaggtcaccactcccaatcctgggggrttggagrggcctggggstgtgggrtgscccatccccctccccatcacctttggtgggggsagggcctg

SEQ ID NO: 13 - protein, 646 aa, Homo sapiens
  VARIANT 579, 598: Xaa = Any Amino Acid
MAEKALEAVGCGLGPGAVAMAVTLEDGAEPPVLTTHLKKVENHITEAQRFSHLPKRSAVDIEFVELSYSVREGPCWRKRGYKTLLKCLSGKFCRRELIGIMGPSGAGKSTFMNILAGYRESGMKGQILVNGRPRELRTFRKMSCYIMQDDMLLPHLTVLEAMMVSANLKLSEKQEVKKELVTEILTALGLMSCSRTRTALLSGGQRKRLAIALELVNNPPVMFFDEPTSGLDSASCFQVVSLMKSLAQGGRTIICTIHQPSAKLFEMFDKLYILSQGQCIFKGVVTNLIPYLKGLGLHCPTYHNPADFIIEVASGEYGDLNPMLFRAVQNGLCAMAEKKSSPEKNEVPAPCPPCPPEVDPIESHTFATSTLTQFCILFKRTFLSILRDTVLTHLRFMSHVVIGVLIGLLYLHIGDDASKVFNNTGCLFFSMLFLMFAALMPTVLTFPLEMAVFMREHLNYWYSLKAYYLAKTMADVPFQVVCPVVYCSIVYWMTGQPAETSRFLLFSALATATALVAQSLGLLIGAASNSLQVATFVGPVTAIPVLLFSGFFVSFKTIPTYLQWSSYLSYVRYGFEGVXLTIYGMERGDLTCLEERCXFREPQSILRALDVEDAKLYMDFLVLGIFFLALRLLAYLVLRYRVKSER

SEQ ID NO: 14 - DNA, 27 bp, Artificial Sequence
  Oligonucleotide used in RT-PCR reaction.
gccaccatggcggagaaggcgctggag

SEQ ID NO: 15 - DNA, 25 bp, Artificial Sequence
  Oligonucleotide used in RT-PCR reaction.
cccacaaatacaactggcatccctg

SEQ ID NO: 16 - DNA, 24 bp, Artificial Sequence
  Oligonucleotide used in RT-PCR reaction.
taccgagctcggatccactagtcc

SEQ ID NO: 17 - DNA, 23 bp, Artificial Sequence
  Oligonucleotide used in RT-PCR reaction.
caggccctgcccccaccaaaggt